(19) United States
(12) Patent Application Publication
(10) Pub. No.: US 2010/0113956 A1
(43) Pub. Date: May 6, 2010

(54) NASAL CANNULA FOR ACQUIRING BREATHING INFORMATION

(75) Inventors: James N. CURTI, Bakersfield, CA (US); James CHUA, Bakersfield, CA (US); Peter W. SALTER, Bakersfield, CA (US)

Correspondence Address: DAVIS & BUJOLD, P.L.L.C., 112 PLEASANT STREET, CONCORD, NH, 03301, US

(73) Assignee: SALTER LABS, Arvin, CA (US)

(21) Appl. No.: 12/035,744

(22) Filed: Feb. 22, 2008

Related U.S. Application Data
(63) Continuation-in-part of application No. 11/155,901, filed on Jun. 17, 2005, now Pat. No. 7,640,932.
Application No. 11/155,901 is a continuation-in-part of application No. 10/627,502, filed on Jul. 25, 2003, now abandoned.
Application No. 10/627,502 is a division of application No. 09/837,720, filed on Apr. 18, 2001, now Pat. No. 6,655,385.
Application No. 09/837,720 is a continuation of application No. 09/184,111, filed on Nov. 2, 1998, now Pat. No. 6,439,234.
Application No. 09/184,111 is a continuation of application No. PCT/US98/05573, filed on Apr. 3, 1998.
(60) Provisional application No. 60/902,935, filed on Feb. 22, 2007.
(60) Provisional application No. 60/045,080, filed on Apr. 29, 1997.

(51) Int. Cl. A61B 5/087

(52) U.S. Cl. 600/538

(57) ABSTRACT

A nasal cannula for monitoring symptoms of sleep apnea and hypopnea, including an elongated main body having a main body chamber and first and second nares to be received within first and second nasal passages of a patient's nose and at least one mouthpiece extending to a patient's mouth. The main body chamber communicates with first and second cannula inlet/outlets and each nare and the mouthpiece includes a gas flow passage extending from the nostril or mouth to the main body chamber. At least one nasal gas flow sensor is located in an gas flow passage of at least one of the nares and at least one oral gas flow sensor located in the mouthpiece gas flow passage, preferably in the regions of the gas flow passages adjacent the main body chamber.

This application is a continuation-in-part of provisional patent application Ser. No. 60/902,935 filed Feb. 22, 2007, which is a continuation-in-part of patent application Ser. No. 11/155,901 filed Jun. 17, 2005, which is a continuation-in-part of patent application Ser. No. 10/627,502 filed Jul. 25, 2003 which is a divisional of patent application Ser. No. 09/837,720 filed Apr. 18, 2001 which is a continuation of patent application Ser. No. 09/184,111 filed Nov. 2, 1998 which is a continuation of International Application PCT/US98/05573 filed Apr. 3, 1998 which claims the benefit of provisional patent application Ser. No. 60/045,080 filed Apr. 29, 1997.

### FIELD OF THE INVENTION

The present invention relates to a cannula having a pair of nares and at least one mouthpiece, each nare and each mouthpiece having a primary aperture or opening formed in an end surface or wall thereof and defining a flow path into and out of the nare or mouthpiece for supplying a desired gas to a nostril of a patient, withdrawing or sampling a desired gas from a nostril or the mouth of a patient, monitoring and detecting changes in the breathing characteristics of a patient by measuring pressure within the nostril or airflow through the mouth during patient breathing, and, in particular, a cannula having an airflow measuring temperature sensor associated each mouthpiece and having either or both of a pressure sensor and an airflow measuring temperature sensor associated with each nare for the purpose of detecting, measuring and diagnosing patient apnea and/or hypopnea.

### BACKGROUND OF THE INVENTION

Sleep apnea and hypopnea are medical terms for breathing irregularities during sleep, such as pauses in breathing, abnormally shallow breathing or a an abnormally slow respiratory rate, and differ in that there is some reduced air flow in a hypopnea episode but a cessation of air flow in an apnea episode. Sleep apnea and hypopnea have over time become to be recognized as significant and relatively common medical problems, the common consequence of which is that the CO₂ level in a patient's blood increases while the oxygen level in the patient's blood decreases proportionately to the severity of the breathing irregularity, which may in turn lead to yet other medical consequences such as brain damage due to lack of oxygen or even death in extreme cases. Other common consequences include disruptive sleep patterns, resulting in increased fatigue, lethargy, decreased ability to concentrate, increased irritability, headaches and other effects of a lack of adequate rest.

The commonly recognized forms of sleep apnea include central and obstructive apnea wherein, in central sleep apnea, also referred to generally as apnea, the brain's respiratory control centers are imbalanced during sleep. Stated simply, the brain includes respiratory mechanisms that control breathing according to the levels of carbon dioxide in the blood and, in central sleep apnea, the neurological feedback mechanism that monitors the levels of blood carbon dioxide does not react quickly enough to maintain an even respiratory rate. As a consequence, the entire system cycles between breathing and not breathing, and the sleeper accordingly stops breathing and then starts again. There is no effort made to breathe during the pause in breathing: there are no chest movements and no struggling.

Obstructive sleep apnea, or hypopnea, is the most common category of sleep-disordered breathing and occurs when breathing is interrupted by a physical block to airflow despite the efforts of the efforts of the respiratory mechanisms to maintain a regular and adequate airflow pattern. Since the muscle tone of the body ordinarily relaxes during sleep, and since, at the level of the throat, the human airway is composed of walls of soft tissue, which can collapse, it is easy to understand why breathing can be obstructed during sleep. Mild, occasional sleep apnea, such as may be experienced during an upper respiratory infection is typically not significant, but chronic, severe obstructive sleep apnea can result in severe complications, including congestive heart failure.

Recognition of the commonality and possible severe effects of sleep apnea and hypopnea by the medical profession has resulted in the development and common use of procedures to test for and detect the various forms of sleep apnea so that appropriate remedial measures and treatments can be applied.

Sleep apnea and hypopnea is conventionally diagnosed by means of an overnight sleep test referred to as a polysomnogram in which a patient's breathing patterns are monitored by apparatus that measures, for example, the levels and changes in air flow or air pressure at the patient's nostrils or mouth during the patient's breathing cycles. The patient's breathing patterns are typically monitored by means of a cannula, that is, a device having a plurality of tubes or passages insertable into the nostrils or located in the region of the mouth to deliver gases to or capture gases from the patients's nose of mouth or both. Cannulas may also be used to measure the air flow and pressures present at the patient's nostrils and mouth by the addition of appropriate sensors to the tubes of the cannula, such as thermistor type sensors to detect air flow and volume by changes in temperature or pressure sensors to detect air flow and volume by the pressure changes resulting from air flow.

It is preferable, and is now required, that the systems used in diagnosis by polysomnogram be able to detect and measure air flows and pressures in both obstructive and central apnea. As discussed above, apnea and hypopnea are distinguished in that hypopnea results in a reduced but continuing air flow while apnea, that is, hypopnea, results in a cessation of air flow, accompanied by a lack of chest movement, and complex apnea is a combination of obstructive apnea and central apnea or hypopnea.

This requirement, however, results in certain design problems in the cannulas to be used in polysomnograms. For example, and as discussed above air flow can be measured at the mouth or at the nostrils, but the difference in characteristics between apnea and hypopnea, that is, a cessation of airflow versus a reduction in air flow, may be confused or misidentified due to differences in the nasal and mouth air passages. For example, air flow measurements by pressure measurement at the nostrils or mouth with be effected by whether the mouth is open or closed. That is, an open mouth will reduce the range of pressure variations at the nostrils while a closed mouth will increase the range of pressure variation at the nose.

It must also be noted that both mouth and nasal measurements for the detection of apnea are subject to yet further problems, and in particular by the response times, sensitivity and possible output signal amplitudes of various types of sensors. For example, pressure sensors have faster response times but generate output signals that are of lower amplitude, so that it is difficult to measure lower levels of air flow. Thermal sensors, such as thermistors and thermocouples, have lower response times but generate higher level signals, thus improving the detection of lower levels of air flow.

### OBJECTS OF THE INVENTION

It is, therefore, an object of the present invention to provide a nasal cannula structure suitable for accurately and reliably identifying the various forms of apnea and for accurately and reliable performing the air flow measurements necessary for polysomnogram diagnosis of the various forms of apnea.

### SUMMARY OF THE INVENTION

The present invention is directed to a nasal cannula for monitoring breathing of a patient and, in particular, a nasal cannula for detecting, measuring and diagnosing symptoms of sleep apnea and hypopnea.

According to the present invention, the cannula includes an elongated main body for positioning adjacent a nose of the patient and having a main body chamber and first and second nares extending from the main body chamber to be received within first and second nasal passages of a patient's nose. The main body chamber communicates with first and second cannula inlet/outlets and each nare includes a gas flow passage extending from an inlet/outlet opening formed in an end surface of the nare and the main body chamber. The cannula further includes at least one mouthpiece having a first end at the main body chamber and a second end positionable at a mouth of the patient and a mouthpiece gas flow passage extending between the main chamber and a mouthpiece inlet/outlet at the second end of the mouthpiece. At least one nasal gas flow sensor is located in an gas flow passage of at least one of the nares and at least one oral gas flow sensor located in the mouthpiece gas flow passage, preferably in the regions of the gas flow passages adjacent the main body chamber.

The cannula may further include a septum dividing the main body chamber into a first main body chamber communicating with the first cannula inlet/outlet and the gas flow passage of one nare and a second main body chamber communicating with the second cannula inlet/outlet and the gas flow passage of the other nare and the mouthpiece gas flow passage may communicate with either of the of the main body chambers. The cannula may also include a second mouthpiece, and each mouthpiece will then communicate with one of the main body chambers.

The nasal gas flow sensors may include a first nasal gas flow sensor located in the gas flow passage of one nare and a second nasal gas flow sensor located in the gas flow passage of the other nare and there may be an oral gas flow sensor located in the gas flow passage of each mouthpiece.

In presently preferred embodiments, there are two nasal gas flow sensors, or which one nasal gas flow sensor is a thermal gas flow sensor and the other is a pressure sensor, and the oral gas flow sensor is a thermal gas flow sensor.

### DETAILED DESCRIPTION OF THE ILLUSTRATED EMBODIMENT

FIG. 1 is a frontal view of a normally positioned nasal cannula on a patient (shown in phantom) connected to a gas source (G) and a gas analyzer (A);

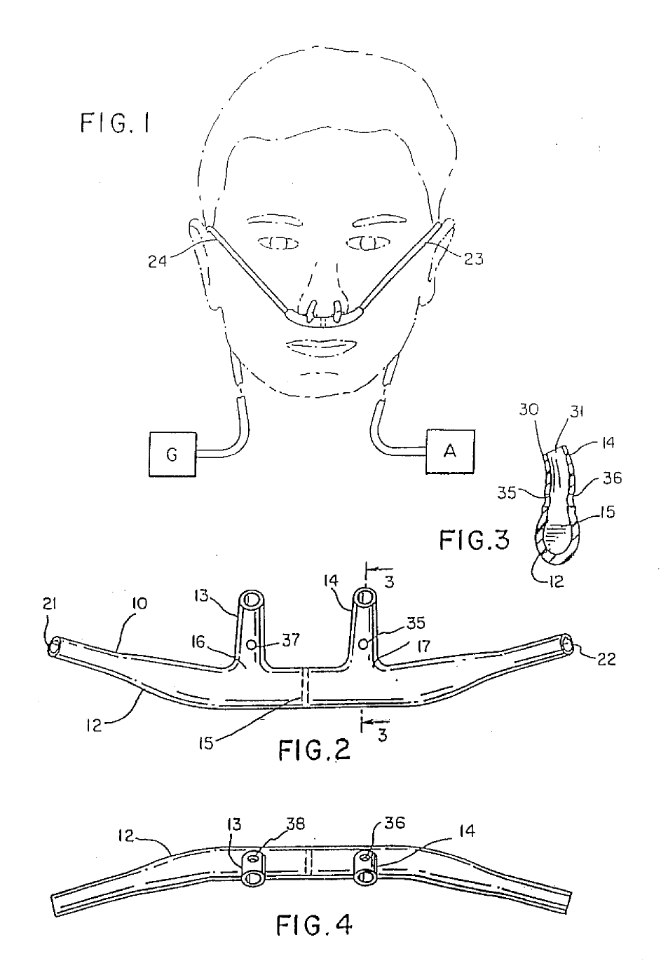

FIG. 2 is a rear view of the cannulas face piece shown in FIG. 1;

FIG. 3 is a partial cross section of a nare of the nasal cannula taken along the lines and arrows 3-3 of FIG. 2;

FIG. 4 is a top plan view of the nasal cannula of FIG. 2;

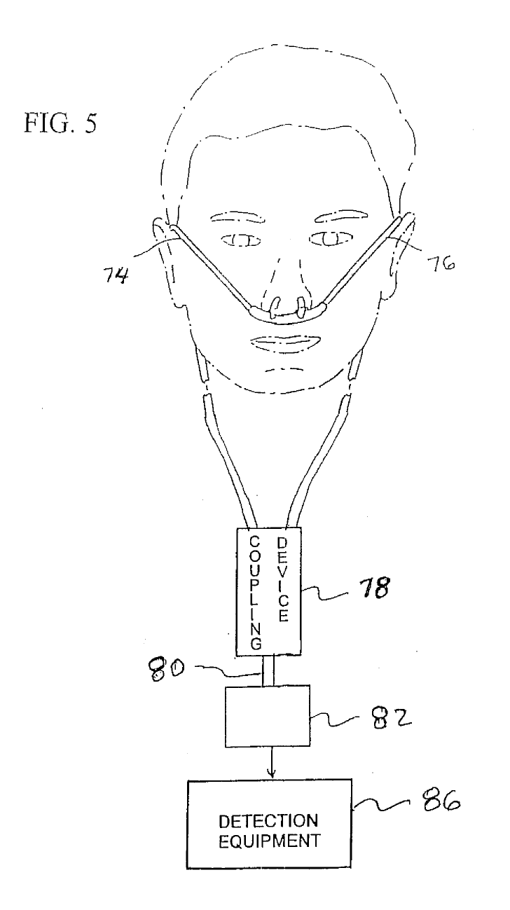

FIG. 5 is a diagrammatic view of a nasal cannula for connection to detection equipment to facilitate monitoring of breathing characteristics of a patient;

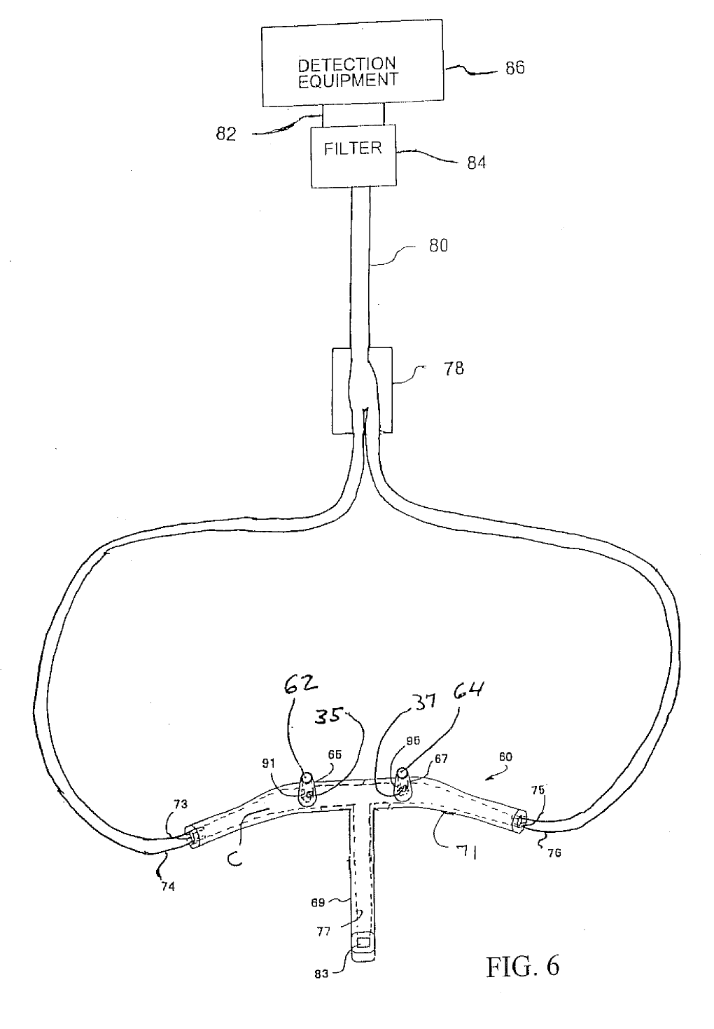

FIG. 6 is a diagrammatic view of a nasal cannula, with a mouthpiece, for connection to detection equipment to facilitate monitoring of breathing characteristics of a patient;

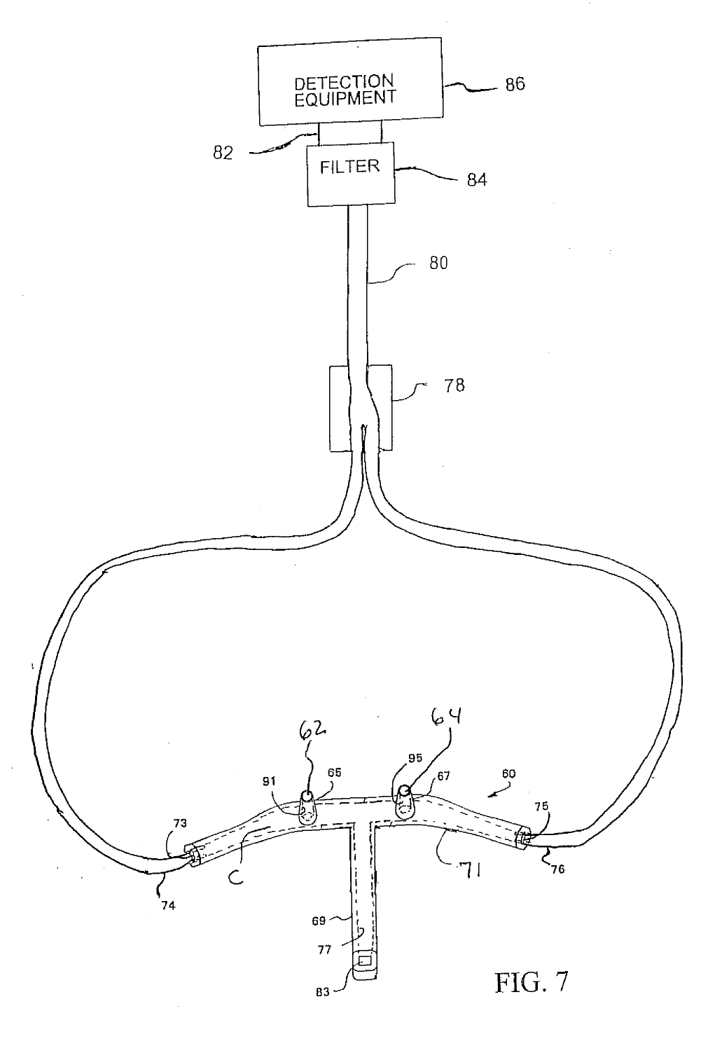

FIG. 7 is a diagrammatic view of a nasal cannula, with a mouthpiece but without any secondary openings in the nares, for connection to detection equipment to facilitate monitoring of breathing characteristics of a patient;

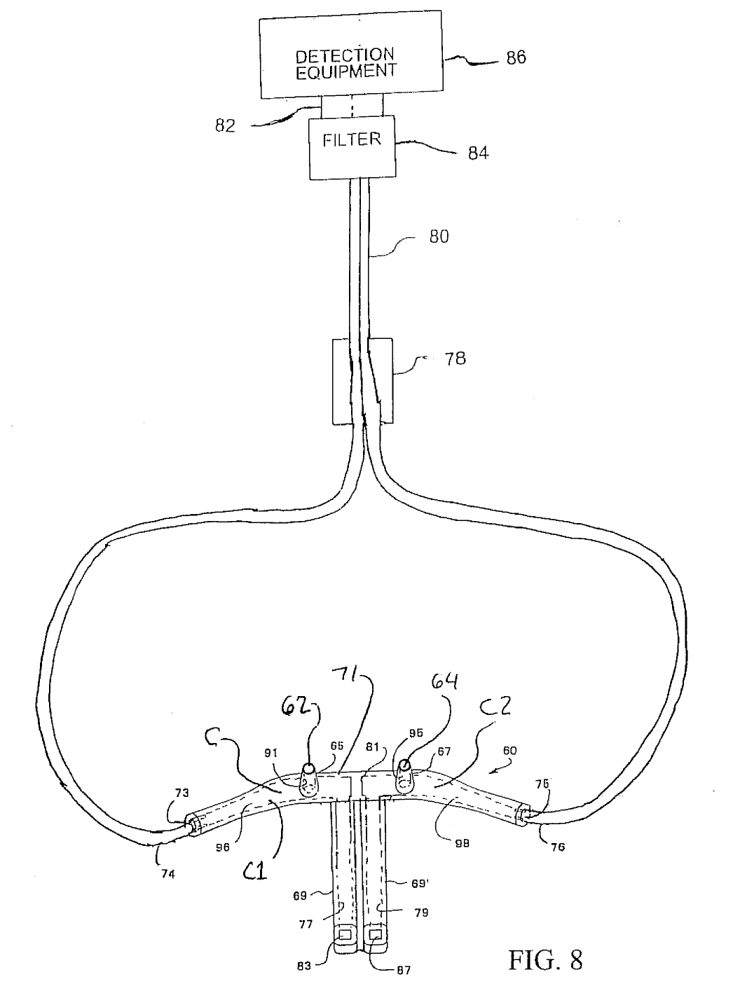

FIG. 8 is a diagrammatic view of a divided nasal cannula, with a pair of integral mouthpieces but without any secondary openings in the nares, for connection to detection equipment to facilitate monitoring of breathing characteristics of a patient;

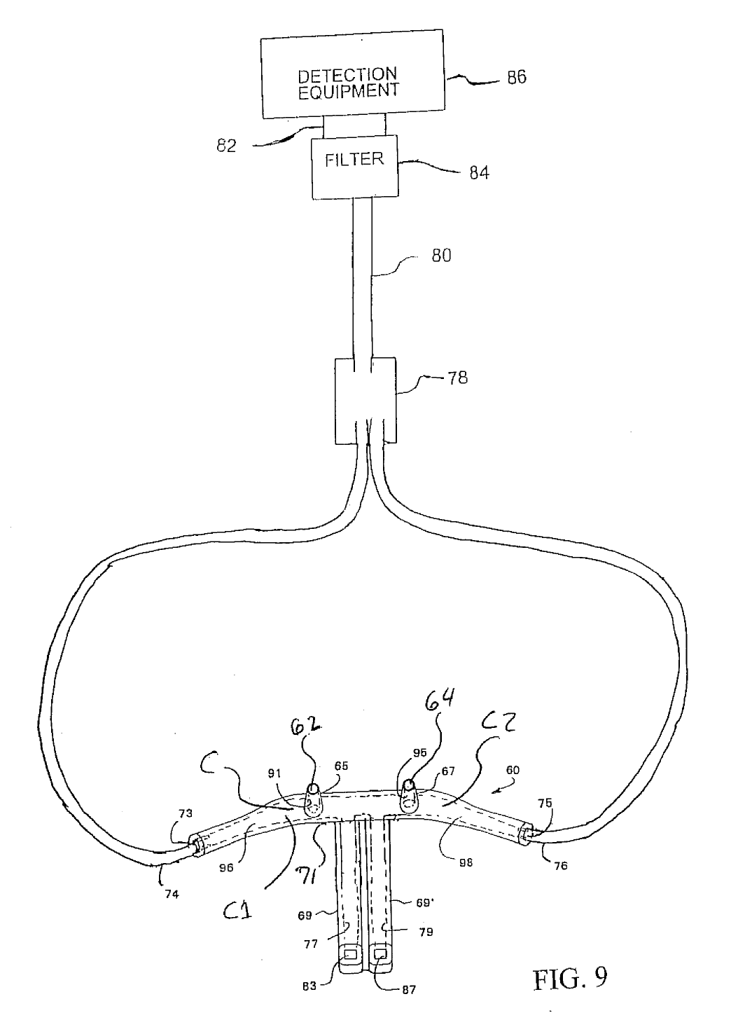

FIG. 9 is a diagrammatic view of an undivided nasal cannula, with a pair of integral mouthpieces but without any secondary openings in the nares, for connection to detection equipment to facilitate monitoring of breathing characteristics of a patient;

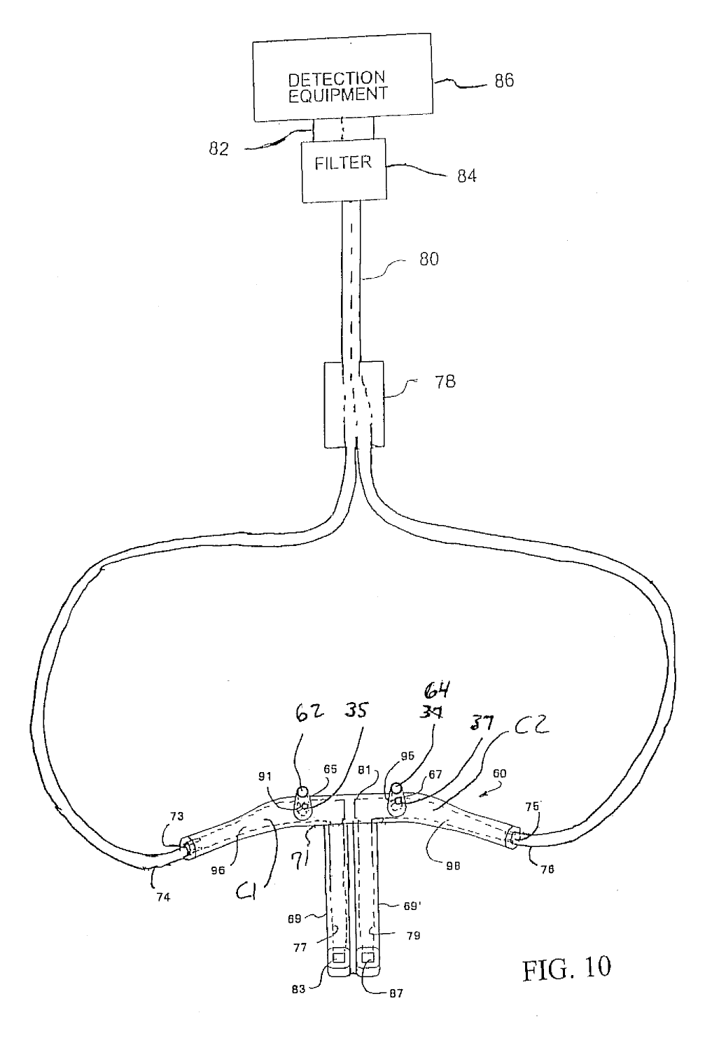

FIG. 10 is a diagrammatic view of a divided nasal cannula, with a pair of mouthpieces and with secondary openings in the nares, for connection to detection equipment to facilitate monitoring of breathing characteristics of a patient;

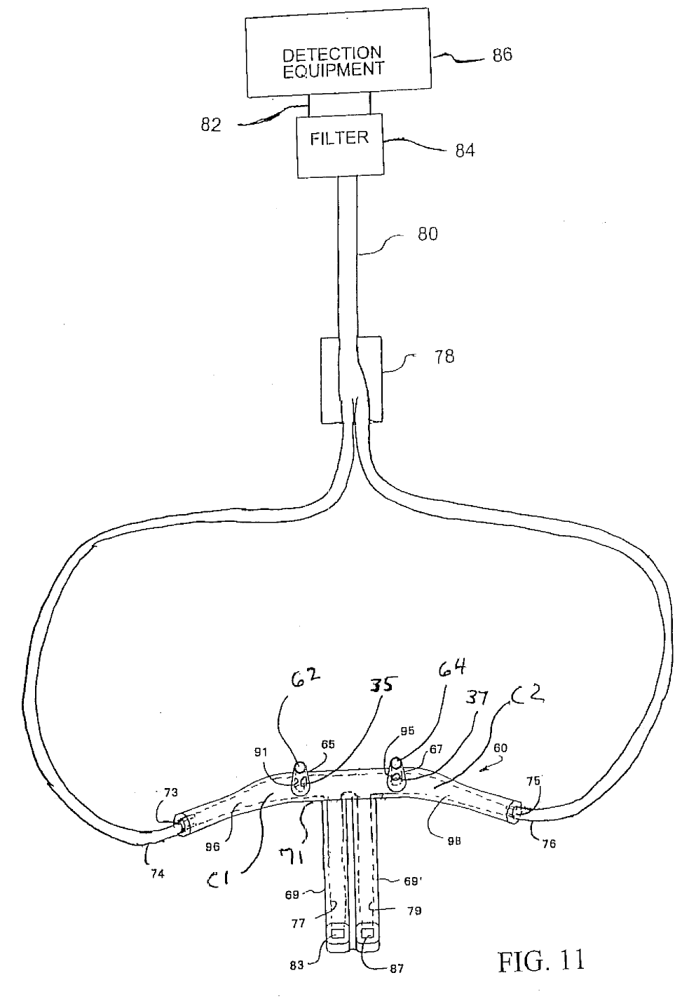

FIG. 11 is a diagrammatic view of an undivided nasal cannula, with a pair of integral mouthpieces and with nares having secondary openings therein, for connection to detection equipment to facilitate monitoring of breathing characteristics of a patient;

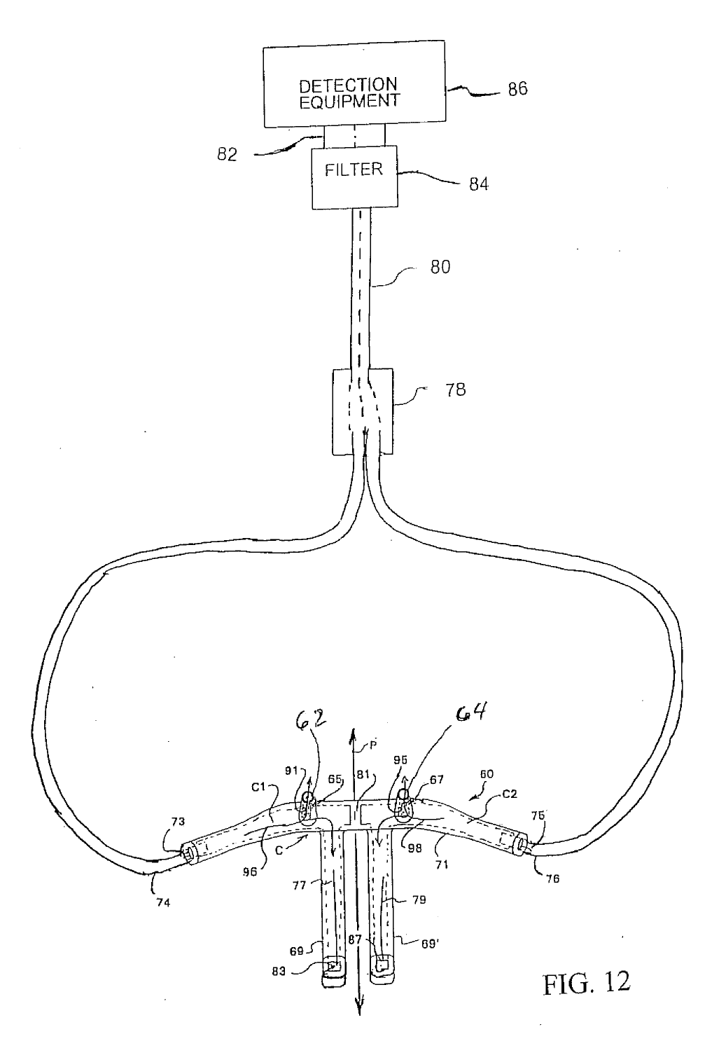

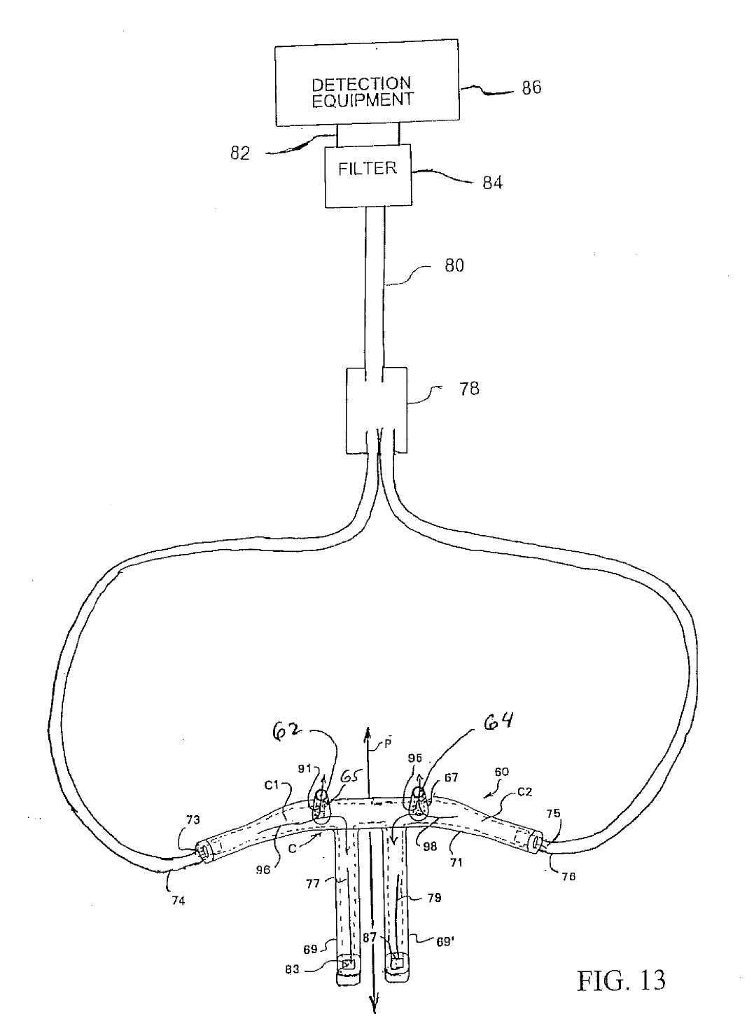

FIG. 12 is a diagrammatic view of a divided nasal cannula, with a pair of separate, spaced apart mouthpieces but without any secondary openings in the nares, for connection to detection equipment to facilitate monitoring of breathing characteristics of a patient;

FIG. 13 is a diagrammatic view of an undivided nasal cannula, with a pair of separate, spaced apart mouthpieces but without any secondary openings in the nares, for connection to detection equipment to facilitate monitoring of breathing characteristics of a patient;

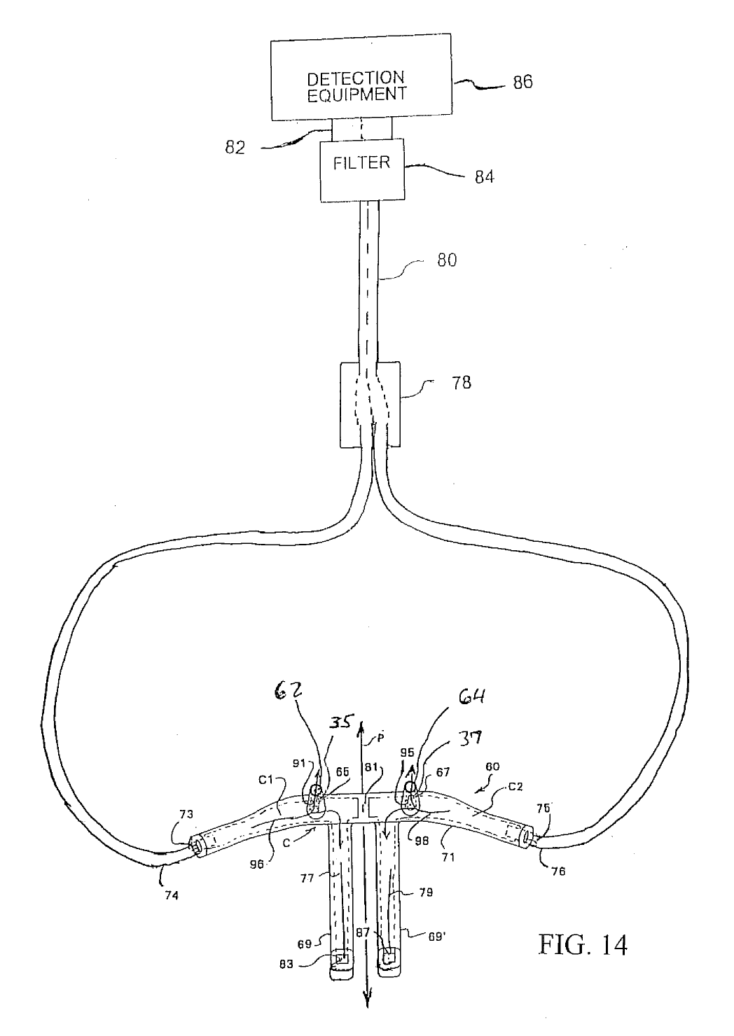

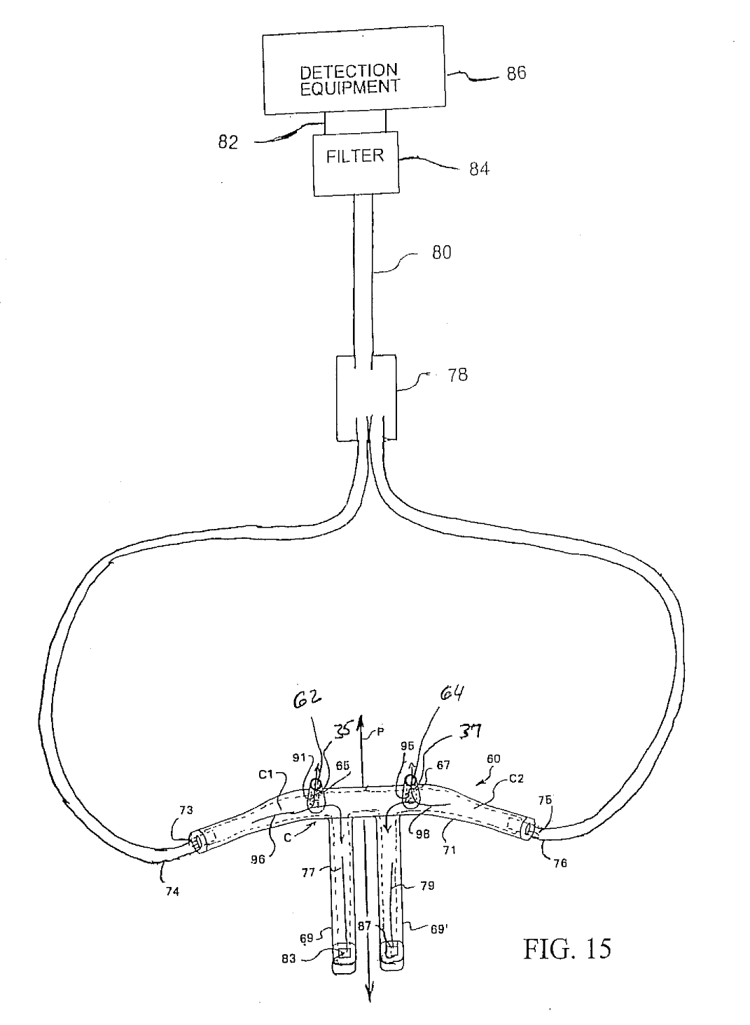

FIG. 14 is a diagrammatic view of a divided nasal cannula, with a pair of separate, spaced apart mouthpieces and with nares having secondary openings therein, for connection to detection equipment to facilitate monitoring of breathing characteristics of a patient;

FIG. 15 is a diagrammatic view of an undivided nasal cannula, with a pair of separate, spaced apart mouthpieces and with nares having secondary openings therein, for connection to detection equipment to facilitate monitoring of breathing characteristics of a patient;

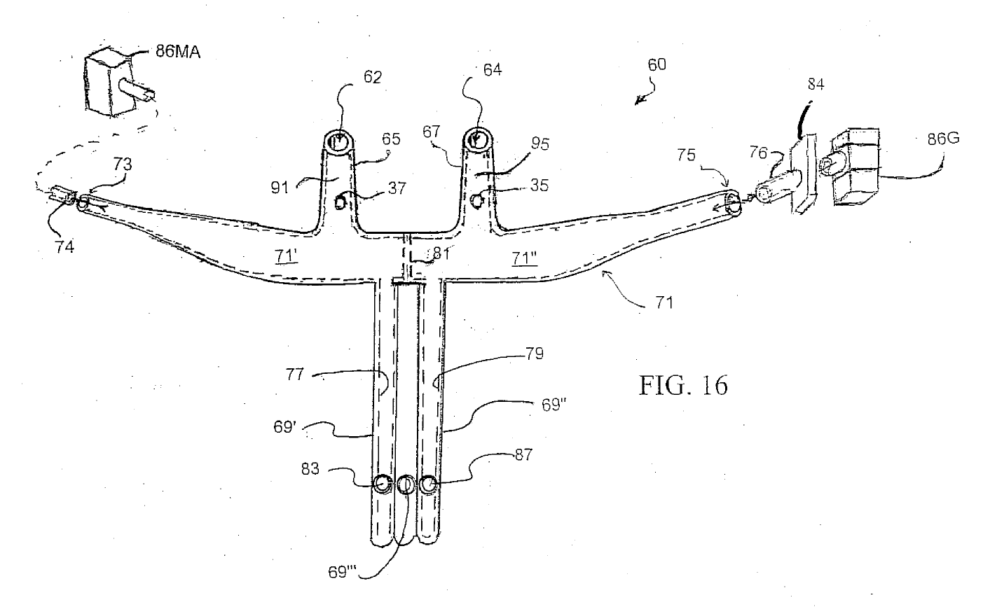

FIG. 16 is a diagrammatic view of a divided nasal cannula, for connection to desired medical equipment, having a pair of separate, spaced apart mouthpieces with each nare has a secondary opening therein;

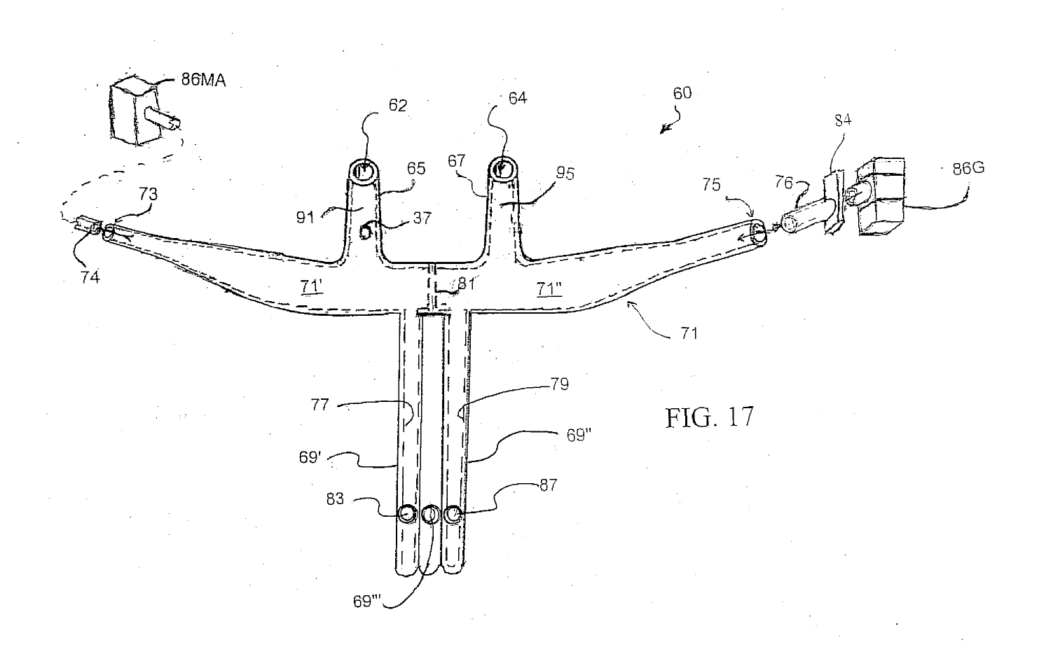

FIG. 17 is a diagrammatic view of a divided nasal, for connection to desired medical equipment, having a pair of separate, spaced apart mouthpieces with only one nare having a secondary opening therein;

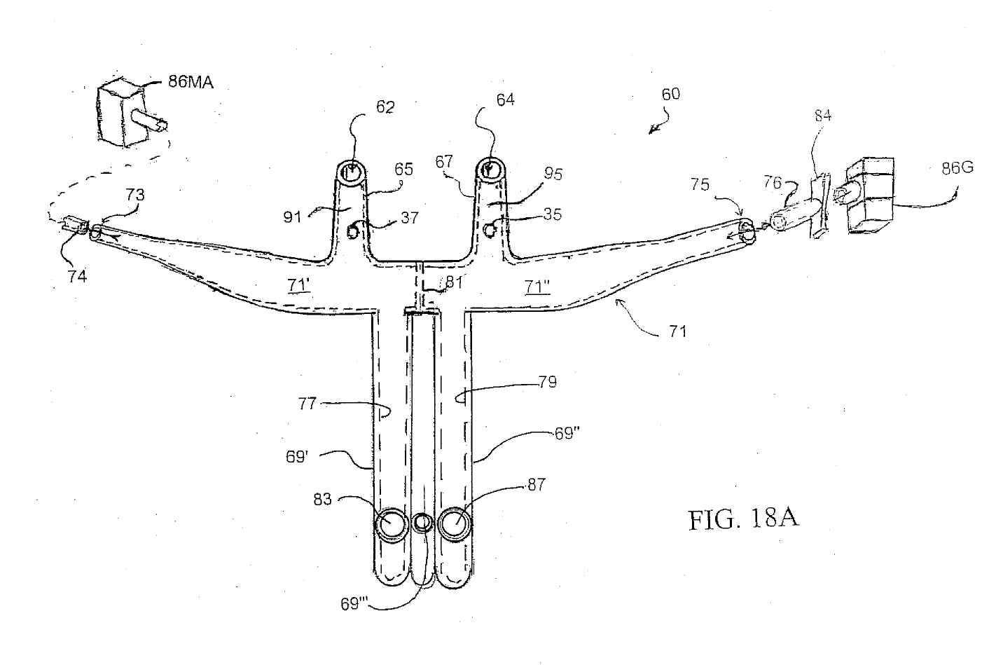

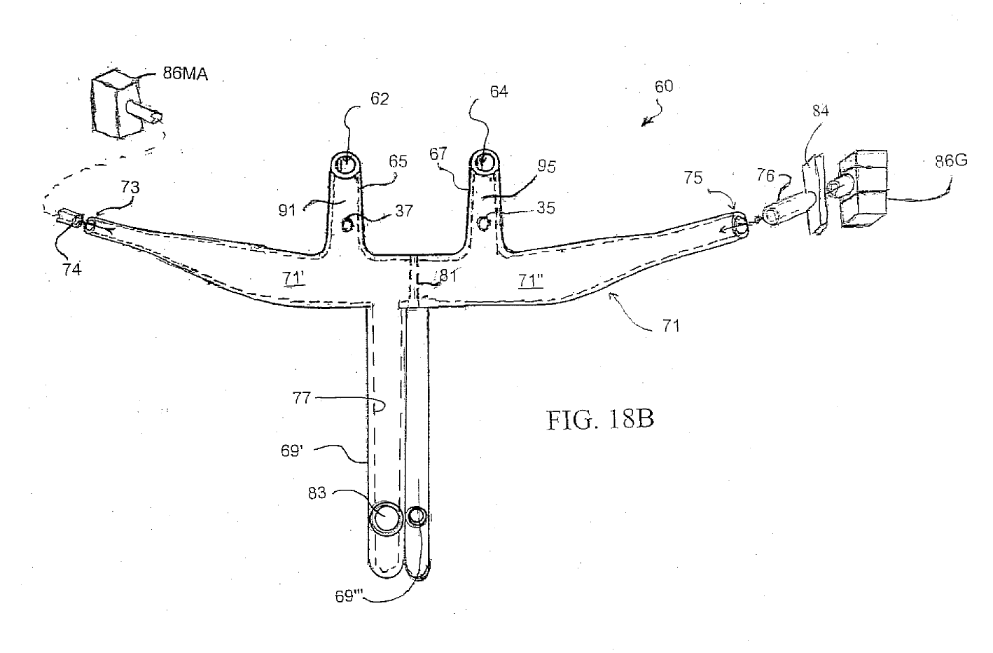

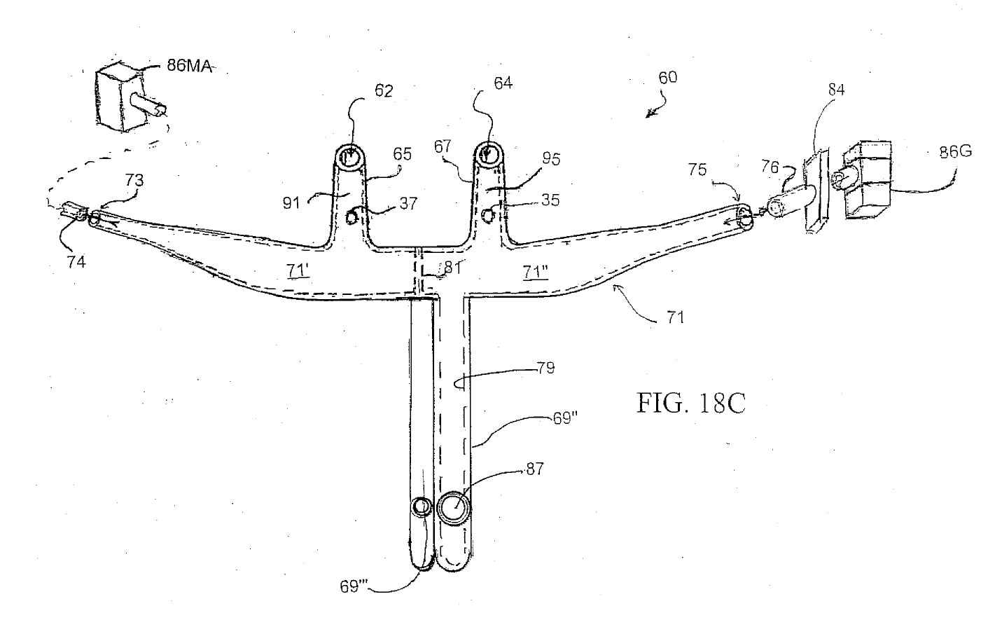

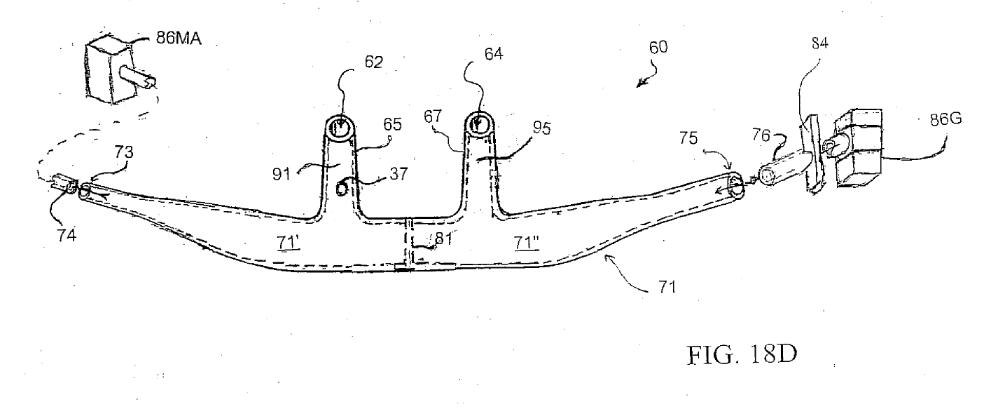

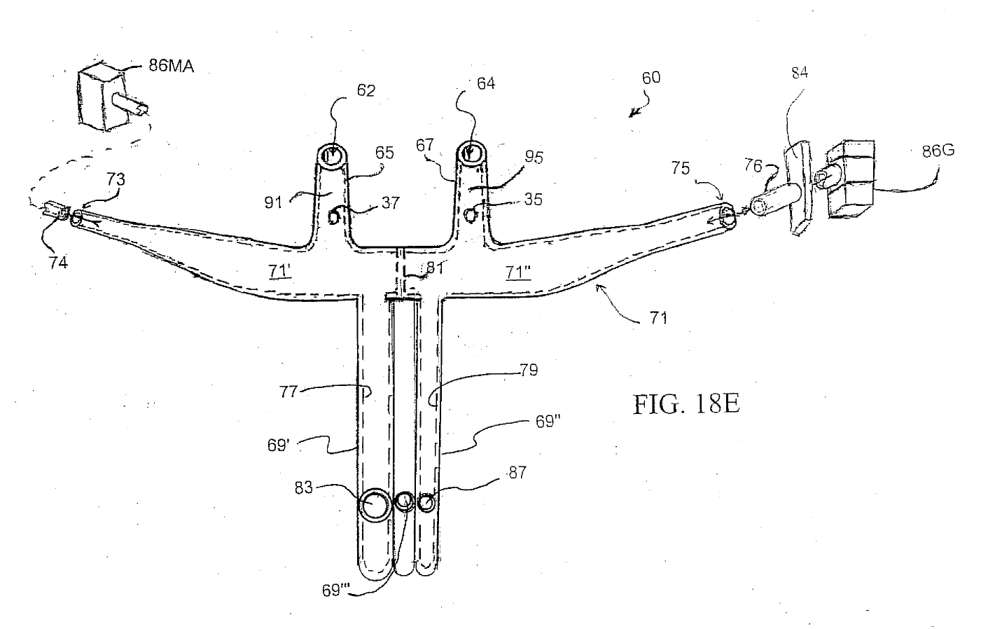

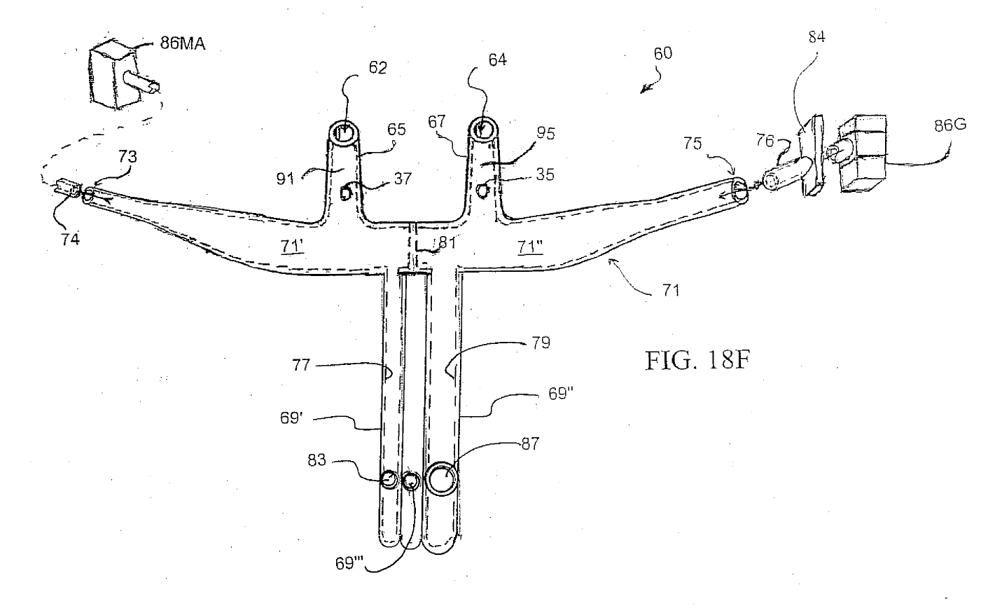

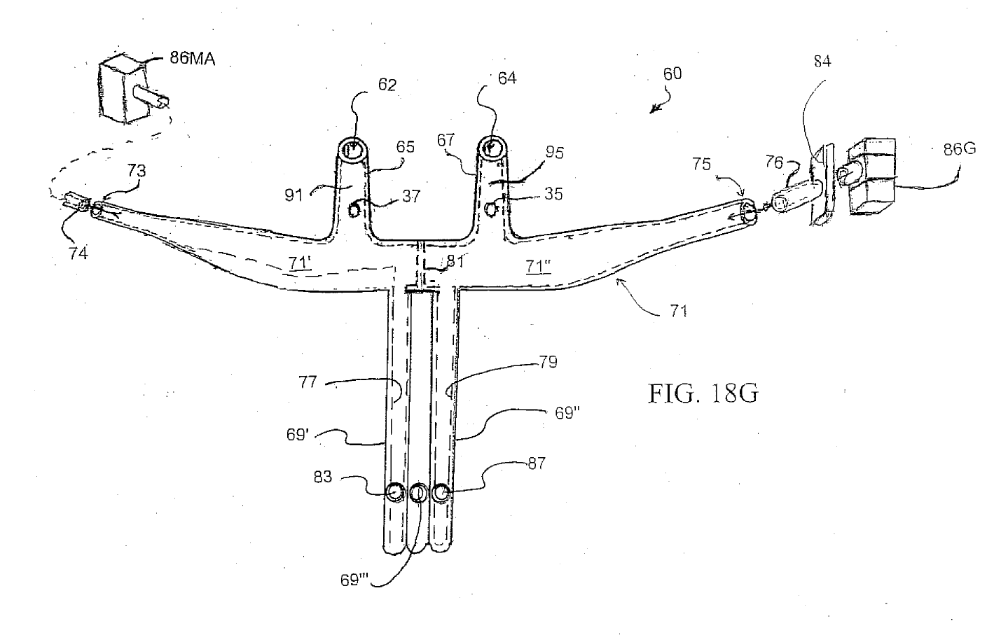

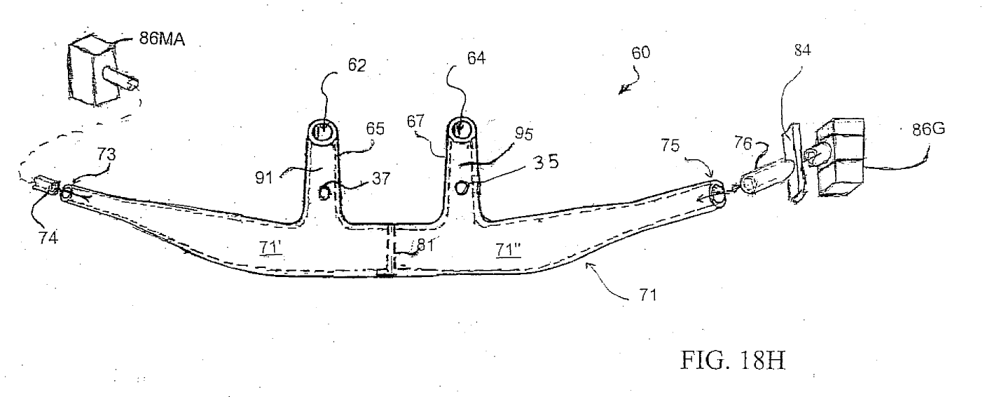

FIG. 18A a diagrammatic view of a divided nasal cannula, according to the present invention, having a pair of spaced apart mouthpieces which both have substantially the same size, relatively large, internal flow passages;

FIG. 18B a diagrammatic view of a divided nasal cannula, according to the present invention, having only a single mouthpiece communicating with one side of the divided cannula;

FIG. 18C a diagrammatic view of a divided nasal cannula, according to the present invention, having only a single mouthpiece communicating with an opposite side of the divided cannula;

FIG. 18D a diagrammatic view of a divided nasal cannula, according to the present invention, without any mouthpiece;

FIG. 18E a diagrammatic view of a divided nasal cannula, according to the present invention, having a pair of spaced apart mouthpieces in which mouthpiece on the left has a larger internal flow passage than the internal flow passage for the mouthpiece on the right;

FIG. 18F a diagrammatic view of a divided nasal cannula, according to the present invention, having a pair of spaced apart mouthpieces in which mouthpiece on the right has a larger internal flow passage than the internal flow passage for the mouthpiece on the left;

FIG. 18G a diagrammatic view of a divided nasal cannula, according to the present invention, having a pair of spaced apart mouthpieces which both have substantially the same size, relatively small, internal flow passages;

FIG. 18H a diagrammatic view of a divided nasal cannula, according to the present invention, without any mouthpiece but with openings in both nares;

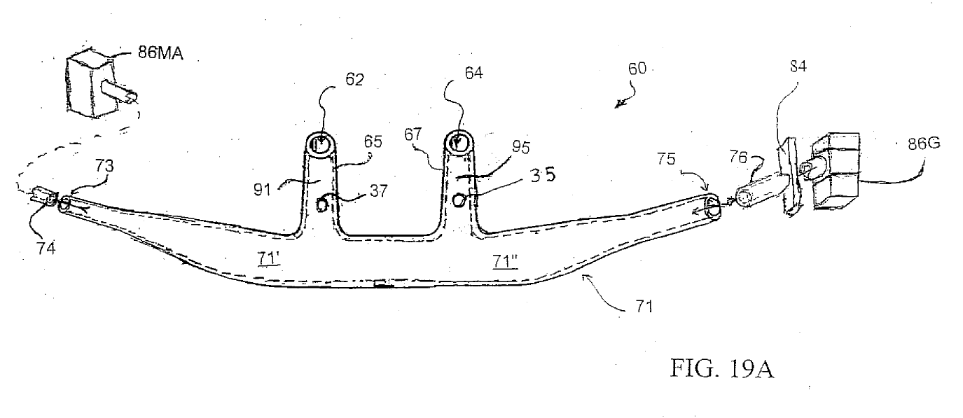

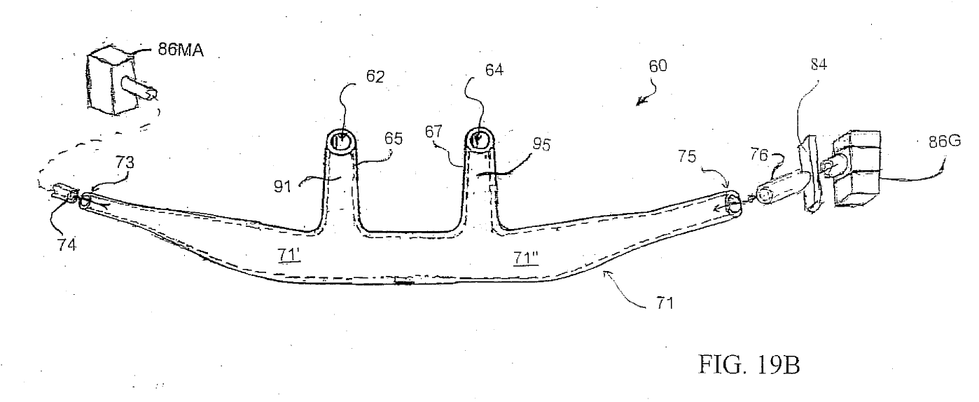

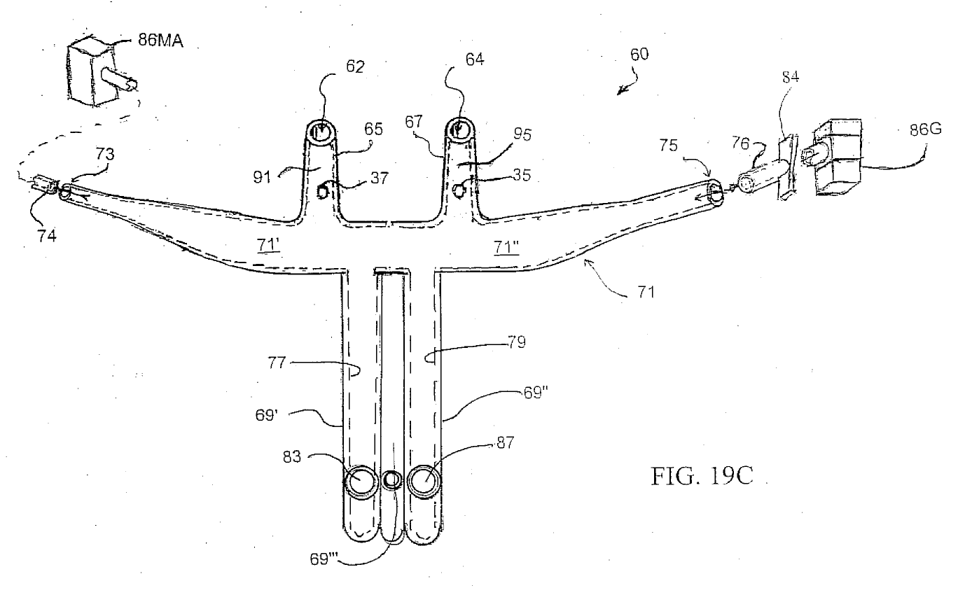

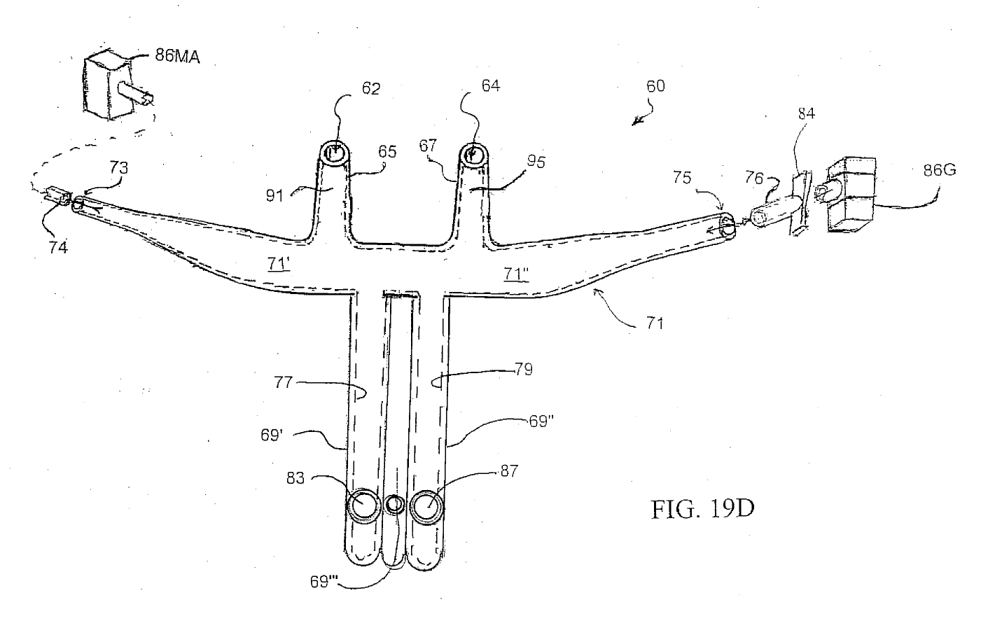

FIG. 19A a diagrammatic view of an undivided nasal cannula, according to the present invention, without any mouthpiece but with openings in the nares;

FIG. 19B a diagrammatic view of an undivided nasal cannula, according to the present invention, without any mouthpiece and without any openings in the nares;

FIG. 19C a diagrammatic view of an undivided nasal cannula, according to the present invention, with a mouthpiece and openings in both of the nares;

FIG. 19D a diagrammatic view of an undivided nasal cannula, according to the present invention, with a mouthpiece and without any openings in the nares; and

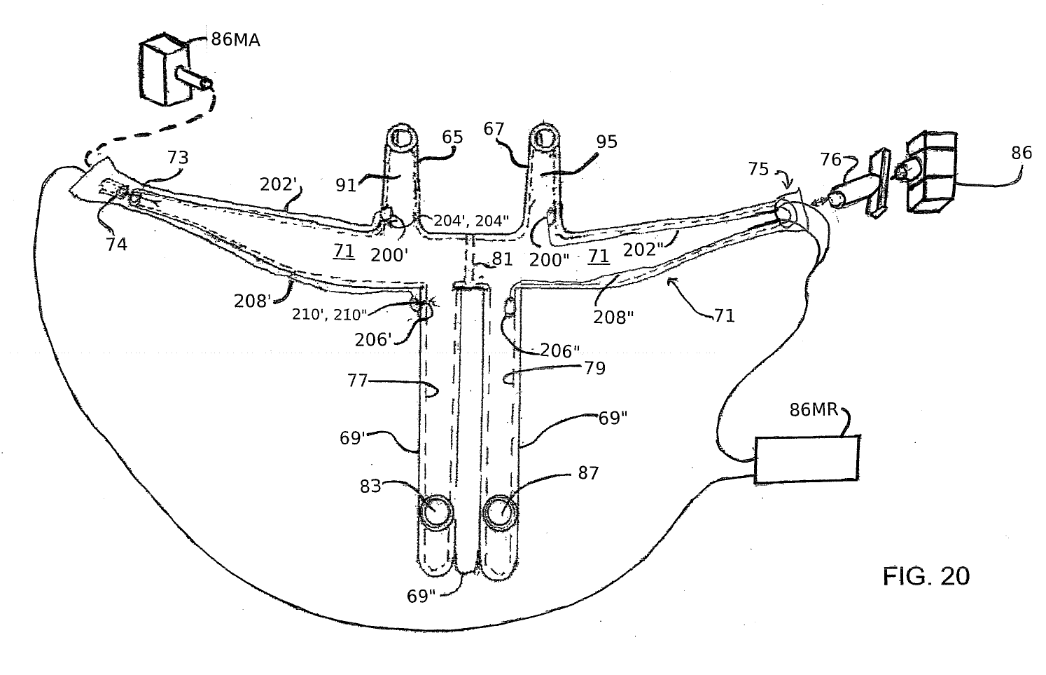

FIG. 20 is a diagrammatic view of a nasal cannula adapted for using in the detection and measurement of sleep apnea.

### DETAILED DESCRIPTION OF THE ILLUSTRATED EMBODIMENT

FIGS. 1-19D and the related text described various embodiments of inventive cannula for various medical purposes and that address various problems of the prior art with regard to such cannula. As described above, the present invention is directed to a cannula specifically adapted for use in the diagnosis of apnea symptoms, such as by polysomnogram, and is described with particular reference to FIG. 20 and the text relating thereto. It will be recognized, however, that the cannula described in and with reference to FIGS. 1-19D may be adapted for use in apnea diagnosis by incorporation of the elements and features of an apnea diagnosis cannula that are described herein below with respect to FIG. 20.

The nasal cannula 10 of one embodiment of the present invention comprises or consists of a generally tubular face piece 12 having two spaced apart nares 13 and 14 and an internal septum 15 disposed in the center of the face piece 12 between the flow passage openings 16 and 17, respectively, of the nares 13 and 14 (see FIGS. 2, 3 and 4). The flow passage openings 21 and 22 on the ends of the face piece 12 are affixed to separate conduits or tubes 23 and 24 as shown in FIG. 1, which are separately connected to a source of insufflating gas (G), such as oxygen, and a commercial carbon dioxide monitoring unit (A) which, in turn, has or is connected to a vacuum pump or other means for drawing an exhaled breath, containing carbon dioxide, into an instrument that is capable of measuring the concentration of the carbon dioxide in the sampled gas.

During use of the cannula for both insufflation and the monitoring of carbon dioxide concentration in the exhaled breath (depicted schematically in FIG. 1), the readings for end-tidal carbon dioxide can become distorted when there is undesirable mixing with room air or with excess insufflating gas. Likewise, carbon dioxide measuring devices which typically employ varying amounts of suction or vacuum to obtain the gas sample to be analyzed, can unduly dilute the sample or more seriously can draw the inlet/outlet opening 31, located in the tip 30 of the sampling nare (representatively shown in FIG. 3), into contact with the adjacent surface of the tissue of the nasal passage and either partially or fully occlude the inlet/outlet opening 31 thereby restricting or even preventing sampling of the exhaled gases for their carbon dioxide concentration.

This is an especially serious problem where the patient is prone to generate secretions and the secretions are present so as to be drawn into the inlet/outlet opening 31 at the tip 30 and then either partially or fully occlude, block, clog or otherwise obstruct the opening 31, during the administration of anesthesia or a sleep diagnostic session, for example.

The anesthesiologist must respond by clearing the nare opening after first removing the cannula from its initially installed location on the face of the patient. This may be complicated especially where the patient is draped in a manner which covers the cannula, such as in eye surgery. It may also be difficult to detect the occlusion where the end-tidal carbon dioxide measurement signal is only partially, but not fully, degraded.

It has been discovered that the expedient of additionally providing the nares with very small holes or openings, shown collectively at 35, 36, 37 and 38 adjacent the tip 30, achieves the desired result of preventing an undesirable and unnecessary level of suction at the opening 31 of the tip 30 from developing sufficiently to draw the opening 31 into the nasal tissue thereby either partially or fully occluding, blocking, clogging or otherwise obstructing the opening 31. The holes are sized large enough to prevent sufficient suction from developing at the tip 30 so as to draw in mucosal secretions or attach the tip by suction to the soft mucosal tissue, while still facilitating drawing an undiluted sample of the exhaled gases to provide good end-tidal carbon dioxide measurements. Likewise, too large an opening for these holes would undesirably dilute the exhaled gas sample with room air or excess insufflation gas.

The openings 35, 36, 37 and/or 38 also facilitate obtaining or withdrawing a desired sample from the nostril (e.g., sampling end tidal CO₂ in the exhaled gases of a patient), monitoring breathing characteristics of a patient (such as respiratory air waves and air flow), or detect changes in pressure within the nostril during patient breathing, etc., by providing a secondary flow path in the event that the primary flow path becomes either partially or fully occluded, blocked, clogged or otherwise obstructed during use of the cannula.

As previously noted, the nasal cannula of the present invention can be used in combination with an oxygen delivery system that delivers the insufflating gas intermittently. The delivery can be initiated at any time after the peak end-tidal carbon dioxide measurement is achieved during exhalation and continuing into the inhalation phase of the breathing cycle or could be inhalation activated or designed to deliver only during selected portions of all or only some of the inhalation phases of a patient's breathing cycles. Preferably, the delivery should begin before the termination of the exhalation phase, such as is described in U.S. Pat. No. 5,626,131. Using intermittent delivery substantially reduces the possibility of distorted carbon dioxide readings clue to gas mixing.

Likewise, slits or slots (not shown) may be employed in the nares which could function in the same manner as the holes describe if they are positioned in such a manner to avoid collapse or occlusion with the nasal tissues and provide the desired function of preventing sufficient suction from developing at the tip of the nare to cause it to be drawn, by suction, onto the tissues. The holes provided as described herein are preferred as there is less risk of occlusion and trauma from the edges of slits or slots to the nasal tissue and potentially there is less risk of occlusion and trauma from the edges of slits or slots to the nasal tissue and potentially there is less risk of gas dilution and mixing from occurring where the slits or slots are overly large.

Further, the combination of intermittent insufflation using the cannula of the present invention produces the desired end-tidal carbon dioxide measurement, as described, and helps prevent patient desaturation during the rigors of surgery and anesthesia administration.

Preferably, the size of the openings from between about 0.05 to about 0.07 of an inch or so though larger or smaller holes or a single hole may be advantageously employed in combination with specific analytical apparatuses. The size and location of the openings can vary with the analyzer selected and the proper function confirmed without undue experimentation.

It is to be appreciated that as discussed above, the cannula may have only a single hollow nasal prong or nare, a pair of nasal prongs or nares and the cannula can be divided or undivided. In addition, the spacing from the nare will vary depending upon whether the cannula is used for neonatal, pediatric or an adult. In addition, the spacing of the mouthpieces, if more than one mouthpiece is utilized, can vary from application from application. The important aspect of the present invention is that the secondary inlet/outlet openings are provided in the nare to allow the nare to function even if the primary inlet/outlet becomes substantially blocked, clogged, obstructed or occluded for one reason or another. The secondary holes allow the nare to still operate and preform the intended function. That is, the secondary inlet/outlet opening(s) still allow the nare to supply a treating gas to the nostril of the patient, allow sampling or withdrawal of an exit gas being exhausted by the nostril of the patient (e.g., sampling end tidal CO₂ in the exhaled gases of a patient), allow monitoring or detection of the breathing characteristic via the nare, etc.

The inventors of the present invention have found that the secondary inlet/outlet opening(s), provided in at least one of the nares, is very useful in monitoring breathing characteristics of a patient (e.g., detecting changes in pressure during breathing), to sleep lab personnel, in the event that one or both of the primary inlet/outlet openings of the nares becomes either partially or fully occluded, blocked, clogged or otherwise obstructed by, for example, mucosal secretions, soft mucosal tissue during use of the nasal cannula, any inhaled particulate matter which may collect within the nostrils, etc.

The inventors have determined that the primary inlet/outlet opening in combination with the secondary inlet/outlet opening(s) ensure the ability of the nare to adequately detect or sense the change in pressure within the nasal cavity of the patient, wearing the nasal cannula, as the patient breathes while he or she is sleeping and being monitored. In addition, the secondary inlet/outlet opening is still able to adequately detect or sense the change in pressure within the nasal cavity of the patient, wearing the nasal cannula, as the patient breathes while he or she is sleeping and being monitored. That is, the secondary inlet/outlet opening is still able to detect changes in pressure, e.g., from negative to positive and vice versa, as the patient discontinues inhalation (negative pressure) and commences exhalation (positive pressure), and vice versa, withdraw or sample a desired gas sample from the nostril (e.g., sampling end tidal CO₂ in the exhaled gases of a patient), monitoring breathing characteristics of a patient (such as respiratory air waves and air flow), etc. As a result of this, the sleep diagnostic equipment is still able to continue monitoring the breathing characteristics of the patient, while the patient remains sleeping.

If the nasal cannula were unable to continue to monitor breathing characteristics of the patient, detect pressure, sample a desired gas, etc., the sleep lab technician will generally alter or manipulate the position of the nasal cannula in an attempt to remove or alleviate the occlusion, blockage, clog or obstruction, while the patient is still sleeping, without waking or arousing the patient. In some instances, the sleep lab technician may have to completely remove the nasal cannula, clear the occlusion, blockage, clog or obstruction, and then reinstall the nasal cannula while the patient is still sleeping, without waking the patient. However, if the patient wakes up during such manipulation or removal by the sleep lab technician, this will delay somewhat the sleep lab test or may possibly cause the sleep lab technician to restart patient testing and this can be costly and time consuming. It is to be appreciated that without the additional secondary inlet/outlet opening, provided in a side wall of the nare, in many instances the patient may be sufficiently aroused or awakened, as the sleep lab technician attempts to manipulate or remove the cannula to remove or alleviate the occlusion, blockage, clog or obstruction.

With reference to FIG. 6, another embodiment of the cannula will now be discussed. As this embodiment is very similar to the previous embodiment, identical reference numerals will given to identical elements and only the differences between this embodiment and the previous embodiment will be discussed in detail.

The only significant difference between this embodiment and the previous embodiment is the addition of a curved mouthpiece which is connected to the main body of the cannula for monitoring the breathing characteristics of a patient, obtaining a desired sample, detecting pressure, for example, for a mouth breathing patient. That is, the nasal cannula 60 comprises a single flow path having three separate inlet/outlet openings 62, 64 and 83 to the central internal chamber or compartment C defined by the main body. Each one of the three inlet/outlet openings 62, 64 and 83 to the central internal chamber or compartment C is suitable for monitoring breathing characteristics, detecting pressure, withdrawing or sampling an exhalation gas(es) from the patient nostril (e.g., sampling end tidal CO₂ in the exhaled gases of a patient), measuring differential air flow along the tubing connect to the cannula, supplying a treating gas to a patient, etc., both via either a nostril or the mouth of a patient. The central chamber or compartment C of the main body 71 of the cannula 60 is in constant and continuous communication with an inlet/outlet opening 83, formed in the end surface of the mouthpiece 69, via a gas passageway 77 in the mouthpiece 69 and also in constant and continuous communication with the inlet/outlet opening 62, formed in the end surface of the first nare 65, and the secondary inlet/outlet opening(s) 35 and/or 36 via a gas passageway 91 in the first nare 65 and in constant and continuous communication with the inlet/outlet opening 64, formed in the end surface of the second nare 67, and the secondary inlet/outlet opening(s) 37 and/or 38 via a gas passageway 95 in the second nare 67. In addition, the central chamber or compartment C of the main body 71 also communicates with first and second opposed chamber end openings 73, 75 of the cannula 60. As a result of this arrangement, each one of these inlet/outlet openings 62, 64 and 83 can facilitate preforming one of the following functions: monitor breathing of a patient via the mouth and/or the nose, sampling the end tidal CO₂ content in the exhaled breath of a patient via the mouth and/or the nose to determine the patient's CO₂ concentration level in the blood, measuring differential air flow along the tubing connect to the cannula, supplying a treating gas to a patient via the mouth and/or the nose, detecting changes in pressure airflow, or detecting apnea via the mouth and/or the nose, etc.

A first conduit or tubing 74 is connected to the first end chamber opening 73 while a first end of a second conduit or tubing 76 is connected to a second chamber end opening 75. The opposed second ends of the first and second conduits or tubings 74 and 76 are connected to a coupling device 78 which couples the first and second conduits or tubings 74 and 76 to a common conduit or tubing 80 which is also connected to the coupling device 78. The opposite end of the common conduit or tubing 80 typically has a leer connector 82 which is either coupled to a filter 84, prior to engaging with a pressure detection device or detection equipment 86 or, preferably, the filter 84 may be incorporated into the conventional luer connector 82 and this unitary structure will then facilitate coupling of the nasal cannula 60 to the pressure detection device or detection equipment 86 in a conventional fashion. The first and second conduits or tubings 74 and 76 each have a length of about 8 inches to about a 24 inches or so and preferably have a length of about 15 to 25 inches or so while the common conduit or tubing 80 typically has a length of about 3 feet to about 10 feet, preferably a length of about 5 to 7 feet or so.

With reference to FIG. 7, another embodiment of the cannula will now be discussed. As this embodiment is very similar to the embodiment of FIG. 7, identical reference numerals will given to identical elements and only the differences between this embodiment and the previous embodiment will be discussed in detail.

The only significant difference between this embodiment and the embodiment of FIG. 7 is the elimination of all of the secondary inlets/outlets openings 35, 36 and 37, 38, adjacent the tip so that each nare 65 and 67 only has a primary flow passage into and out of the nare but not any secondary flow passage in the event that the primary inlets/outlets openings 62, 64 of the nares 65, 67 become either partially or fully occluded, blocked, clogged or otherwise obstructed during use of the nasal cannula. That is, flow into and out of the nares 65, 67 can only occur via the primary inlet/outlet openings 62, 64 formed in the nares 65, 67, respectively.

With reference to FIG. 8, still another embodiment of the cannula will now be discussed. As this embodiment is very similar to the embodiment of FIG. 7, identical reference numerals will given to identical elements and only the differences between this embodiment and the previous embodiment will be discussed in detail.

The only significant differences between this embodiment and the embodiment of FIG. 7 is the inclusion of a second integral mouthpiece 69′ and the addition of an internal divider or septum 81, within the internal chamber or compartment C, which divides the internal area of the cannula into two separate chambers or compartments C1 and C2. That is, the nasal cannula 60 comprises two completely separate internal flow paths 208 and 98. Each one of the two completely separate internal flow paths 208 and 98 is suitable for monitoring breathing characteristics, detecting pressure, withdrawing or sampling an exhalation gas(es) from the patient nostril (e.g., sampling end tidal CO₂ in the exhaled gases of a patient), measuring differential air flow along the tubing connect to the cannula, supplying a treating gas to a patient, etc., both via a nostril and the mouth of a patient. The first compartment or passageway C1, of the internal chamber C of the main body 71 of the cannula 60, is in constant and continuous communication with an inlet/outlet opening 83, formed in the end surface of the first mouthpiece 69, via a gas passageway 77 in the first mouthpiece 69 and also in constant and continuous communication with the inlet/outlet opening 62, formed in the end surface of the first nare 65, via a gas passageway 91 in the first nare 65 and with the first chamber end opening 73 of the cannula and all of these components and passageways form the first completely separate internal flow path 208.

The second compartment or passageway C2, of the internal chamber C of the main body 71 of the cannula 60, is in constant and continuous communication with the inlet/outlet opening 87, formed in the end surface of the second mouthpiece 69′, via a gas passageway 79 in the second mouthpiece 69′ and also in constant and continuous communication with the inlet/outlet opening 64, formed in the end surface of the second nare 67, via a gas passageway 95 in the second nare 67 and with the second chamber end opening 75 of the cannula 60 and all of these components and passageways, form the second completely separate internal flow path 98. As a result of these completely separate fluid passageways 208, 98, each completely separate fluid passageway 208 or 98 can facilitate preforming one of the following functions: monitor breathing of a patient via the mouth and/or the nose, sampling the end tidal CO₂ content in the exhaled breath of a patient via the mouth and/or the nose to determine the patient's CO₂ concentration level in the blood, measuring differential air flow along the tubing connected to the cannula, supplying a treating gas to a patient via the mouth and/or the nose, detecting apnea via the mouth and/or the nose, etc. If desired, the septum 81 may be eliminated (as in FIG. 9) so that the first and second compartments or passageways C1 and C2, the first and second internal gas passageways 77, 79 and the first and second gas passageways 91 and 95 in the nares 65 and 67 and all of the openings 62, 64, 73, 75, 83 and 87, are in constant and continuous communication with one another.

With reference to FIG. 10, yet another embodiment of the cannula will now be discussed. As this embodiment is very similar to the embodiment of FIG. 8, identical reference numerals will given to identical elements and only the differences between this embodiment and the previous embodiment will be discussed in detail.

The only significant difference between this embodiment and the embodiment of FIG. 8 is the inclusion of at least one, and preferably a pair of secondary inlets/outlets openings 35, 36 and 37, 38, adjacent the tip of each one of the nares 65, 67 to provide a pair of secondary flow passages 208, 98 in the event that the primary inlets/outlets openings 62, 64 of the nares 65, 67, respectively, become either partially or fully occluded, blocked, clogged or otherwise obstructed during use of the nasal cannula 60. The secondary inlets/outlets openings 35, 36 and 37, 38 are smaller than the primary inlet/outlet openings 62, 64 but are large enough to facilitate withdrawing or sampling a desired gas sample from the nostril (e.g., sampling of end tidal CO₂ in a patient), monitoring breathing characteristics of a patient (such as respiratory air waves and air flow), detecting changes in pressure within the nostril, etc., during patient breathing.

With reference to FIG. 11, yet another embodiment of the cannula will now be discussed. As this embodiment is very similar to the embodiment of FIG. 10, identical reference numerals are given to identical elements and only the differences between this embodiment and the previous embodiment will be discussed in detail.

The only significant difference between this embodiment of FIG. 11 and the embodiment of FIG. 10 is that the septum 81 is eliminated so that the first and second compartments or passageways C1 and C2, the first and second internal gas passageways 77, 79 and the first and second gas passageways 91 and 95 in the nares 65 and 67 and all of the openings 35, 36, 37, 38, 62, 64, 73, 75, 83 and 87 are in constant and continuous communication with one another.

With respect to the embodiments of FIGS. 8-11, it is to be appreciated that it is not necessary to have the first and second mouthpieces 69, 69′ precisely centered between the nares 65, 67. It is possible to position the first and second mouthpieces on one side or the other of a central plane P bisecting a center of main body 71 into two halves. Alternatively, the first and second mouthpieces 69, 69′ could be spaced apart from one another and formed as two completely separate and curved mouthpieces (as shown in FIGS. 12-15) with a septum 81 dividing the internal chamber or compartment into two separate internal flow paths 208 and 98 (as shown in FIGS. 12 and 14). Each one of the two completely separate internal flow paths 208 and 98 is suitable for monitoring breathing characteristics, detecting pressure, withdrawing or sampling an exhalation gas(es) from the patient nostril (e.g., sampling end tidal CO₂ in the exhaled gases of a patient), supplying a desired gas to the patient, etc.

In addition, the first and second mouthpieces could be spaced apart from one another, without having a dividing septum 81 therebetween (as shown in FIG. 13), so that both of the nares 65, 67 and both of the first and second mouthpieces 69, 69′ all communicate with one another via the common central chamber or compartment formed within the main body 71 of the cannula 60. The spacing of the mouthpieces from one another by a distance of from about 0.25 to about 1.25 inches, and more preferably about 0.5 to about 1.0 inches, is useful if the patient being monitored tends to breath, when mouth breathing, out of one side of his or her mouth. By spacing the mouthpieces from one another, mouthpieces of the nasal cannula are better positioned to still detect or monitor breathing of the patient.

In addition, the first and second nares 65, 67 of FIGS. 12 and 13 may be each provided with at least one, and preferably a pair of secondary inlets/outlets openings, 35, 36 and 37, 38, adjacent the tip 30 of the nares to provide a pair of secondary flow passages 208, 98 (as shown in FIGS. 14 and 15, respectively) in the event that the primary inlets/outlets openings 31 of the nares 65, 67 become either partially or fully occluded, blocked, clogged or otherwise obstructed during use of the nasal cannula. The secondary inlets/outlets openings, 35, 36 and 37, 38 are smaller than the primary inlet/outlet opening but are large enough to facilitate withdrawing or sampling a desired gas sample from the nostril (e.g., sampling of end tidal CO₂ in a patient), monitoring breathing characteristics of a patient (such as respiratory air waves and air flow), detecting changes in pressure within the nostril, etc., during patient breathing.

If desired, one or both of the mouthpieces 69, 69′ of the cannula 60 can be provided with a shape retaining, dead soft material or wire (not shown) to facilitate alignment and retention of the first and/or second mouthpieces 69, 69′ in a desired aligned position during use of the cannula 60. The wire permits the mouthpiece 69 or 69′ to be bent, configured or molded into a desired shape, configuration or position while still retaining such desired shape, configuration or position following adjustment of the mouthpiece 69 and/or 69′. A copper wire (either insulated or uninsulated), for example, has substantially no structural memory of any previous shape, orientation, configuration or form which would cause the wire to retain, return or spring back to such previous shape, orientation, configuration or form. Copper is a highly malleable metal and generally retains whatever shape is imparted thereto at any particular time without reverting or returning back to any prior or previous shape. Copper is also a preferred dead soft material, over for example iron, steel or other ferromagnetic materials, due to the propensity of the nasal cannula to be used in connection with a patient exposed to certain electromagnetic and magnetic environments and/or diagnosis procedures.

The wire can either be formed integral with the first and/or second mouthpieces 69, 69′, can be accommodated within an integral compartment extending along the length of the first and/or second mouthpieces 69 and/or 69′, or can be glued or otherwise permanently secured or affixed to an exterior surface of the first and/or second mouthpieces 69 and/or 69′, along the entire length thereof, so that the wire does not become separated or dislodged from the cannula during use of the nasal cannula. The wire typically has a diameter of between 0.01 and 0.2 inches or so.

The first and second mouthpieces 69, 69′ each have a radius of curvature of between about 0.5 of an inch to about 2.5 inches or so, and more preferably a radius of curvature of between about 0.75 of an inch to about 1.25 inches or so. The radius of curvature of the mouthpieces 69, 69′ can vary, depending upon the cannula being manufactured and/or its application, but is generally chosen to facilitate the alignment of an opening formed in the free end of the mouthpiece 69 and/or 69′ with the opening of a mouth of the patient. The first and second mouthpieces 69, 69′ each define an internal passageway 77, 79 therein which has a transverse cross sectional flow area of between about 0.006 and about 0.007 square inches.

Having described above the basic aspects and features of a nasal cannula, according to the present invention, the following description will describe further aspects, features and implementations of the present invention, employing the same reference numerals where appropriate. Such further embodiments of the above described nasal cannulas are illustrated in FIGS. 16, 17, 18A through 18H and 19A-19D and described below.

As illustrated in FIGS. 16 and 17, the nasal cannula 60 of the present invention includes a generally tubular hollow main body 71 that is, in each of these embodiments, internally divided by a partition, a divider or a septum 81 into a first main body chamber 71′ and a second main body chamber 71″. As discussed herein above, the first and the second main body chambers 71′ and 71″ each provide separate gas flow paths between the patient and desired ones of various gas sources and measuring and analysis devices, as discussed herein above.

For example, as shown in FIGS. 16 and 17 and as discussed herein above, the first main body chamber 71′ and the first chamber end opening 73 typically serve as an output path from the patient and through a first tube 74 to, for example, a capnagraph device or a gas monitoring/analysis device 86MA, also as discussed herein above. The second main body chamber 71″ and the second chamber end opening 75, in turn, typically serve as an input or supply path to the patient from, for example, a source 86G for an insufflating gas through a second tube 76 that is connected to second chamber end opening 75.

As with the previous embodiments a section of a first conduit or tubing 74 is connected to the first end chamber opening 73 while a section of a first end of a second conduit or tubing 76 is connected to a second chamber end opening 75. The opposed second ends of the first section of the first and the second conduits or tubings 74 and 76 are typically joined with one another by a conventional coupling device 78 and a second section of the first and the second conduits or tubings 74′, 76′ extends from the coupling device 78 to the desired equipment, apparatus or device 86MA or 86G. The opposite end of the second section of each of the first and the second conduits or tubings 74′, 76′ typically has a luer connector 82′ (e.g., either male of female) which may be either coupled to a filter 84, prior to engaging with the equipment, apparatus or device 86MA or 86G or may be directly couple to the equipment, apparatus or device 86MA or 86G. If a filter is utilized, preferably the filter 84 is incorporated into the conventional luer connector 82 and this unitary structure facilitates coupling of the nasal cannula 60 to the equipment, apparatus or device 86MA or 86G in a conventional fashion. The first section of each of the first and the second conduits or tubings 74 and 76 each have a total length of about 8 inches to about a 24 inches or so and preferably have a length of about 15 to 25 inches or so while the second sections of each of the first and the second conduits or tubings 74′, 76′ each typically have a length of about 3 feet to about 10 feet, preferably a length of about 5 to 7 feet or so.

As also discussed herein above with reference to FIGS. 1-15, the nasal cannula 60, according to the present invention, provides separate gas flow paths between one of the first and the second main body chambers 71′ and 71″ and a desired one of the nasal passages and the mouth of a patient. For example, and as described herein above, the flow passages between the first and the second main body chambers 71′ and 71″ and the nasal passages of the patient are provided by the first and the second nares 65 and 67. The flow path between the first main body chamber 71′ and a first nasal passage of the patient is formed by the internal flow passage 91 through the first nare 65 and the first nare end opening 62 in the first nare 65, while the flow path between the second main body chamber 71″ and the second nasal passage of the patient is formed by the internal flow passage 95 through the second nare 67 and the second nare end opening 64 in second nare 67. In this regard, it will also be understood that the flow path 95 through the second nare 67 typically conveys a flow of insufflating gas, e.g., oxygen, from the gas source 86G to the patient, while the flow path 91, through the first nare 65, typically conveys a gas sample(s) from the patient to the gas monitoring/analysis devices 86MA.

As also shown in FIGS. 16 and 17, and as discussed above with reference to FIGS. 1-15, either or both of the nares 65 and 67 may be provided with one or more alternate flow path holes or openings 35, 37 which are generally located or spaced further from the nare end openings 62, 64 and located slightly closer to the main body 71 of the cannula 60. If two alternate flow path openings 35, 37 are provided in a nare 65 or 67, then such flow path openings 35 or 37 are generally aligned with one another and form a through hole which extends completely through the side wall of the nare 65 or 67. As described, the alternate flow path openings 35, 37 prevents the occurrence of an undesirable and/or unnecessary level of suction at the nare end openings 62, 64 which might otherwise cause either of the nare end openings 62, 64 to become partially or fully occluded, blocked, clogged or otherwise obstructed during utilization of the cannula 60. As described herein above, the alternate flow path openings 35, 37 are sized sufficiently large enough to prevent the development, at the nare end openings 62, 64, of ample suction that could draw in mucosal secretions from the patient or that would draw and/or otherwise force the tips of nares 65, 67 against the soft mucosal tissue of the nostril of a patient by suction. In this regard, it must also be noted, however, that if the alternate flow path openings 35, 37 are too large, then the exhaled gas sample(s) could become undesirably or excessively diluted with, for example, room air and/or possibly excess insufflation gas.

It must also be noted that the alternate flow path openings 35, 37 provide one or more secondary flow paths, between the patient's nostrils and the first and the second main body chambers 71′ and 71″ of the cannula, in the event that either of the primary flow paths, which includes the nare end openings 62, 64, become either partially or fully occluded, blocked, clogged or otherwise obstructed during use. The alternate flow path opening(s) 35, 37 thereby facilitates obtaining or drawing a sample of gas from the patient's nostril, e.g., sampling the end tidal CO₂ in the exhaled gases of a patient, monitoring breathing characteristics of a patient, such as the patient's respiratory air waves and air flow, detecting changes in pressure within the patient's nostril during patient breathing, and so forth, in the event that either of the nare end openings 62, 64 become partially or completely obstructed.

Lastly with regard to the nares 65, 67, and as illustrated in FIGS. 16 and 17 and as will be discussed in detail below concerning the alternate flow path openings 35, 37, it must be noted that either or both of the nares 65, 67 may not be provided with an alternate flow path opening(s) 35, 37 for the reasons discussed herein below.

Further flow paths, between the first and the second main body chambers 71′ and 71″ and a patient's mouth, are also provided by first and second mouthpieces 69′ and 69″ which, as described herein above, are essentially integral hollow tubes extending in a curved configuration downward from the main body 71 and communicating with either the first or the second main body chambers 71′ and 71″ and terminating or ending at or in the region in front of the patient's mouth. The first mouthpiece 69′, for example, includes a first internal flow path 77 communicating with and extending from the first main body chamber 71′ and terminating in a mouthpiece end opening 83 which is positionable adjacent an open mouth of the patient. The second mouthpiece 69″, in turn, includes a second internal flow path 79 communicating with and extending from the second main body chamber 71″ and terminating in a mouthpiece end opening 87 which is positionable adjacent an open mouth of the patient. It will be understood that the second mouthpiece 69″ typically provides a flow path, for example, for the insufflating gas to the patient while the first mouthpiece 69′ typically provides a flow path by which a gas sample, for example, may be retrieved from the patient's exhaled breath and conveyed to the gas monitoring/analysis devices 86MA.

As also discussed herein above, one or both of the mouthpieces 69′, 69″ of the cannula 60 can be provided with a shape forming and retaining element 69″ that may comprise, for example, a shape retaining, dead soft pliable material, element, component or wire, such as copper, to facilitate desired alignment and/or retention of the first and/or the second mouthpieces 69′, 69″ in a desired aligned orientation or position, relative to the patient's mouth during use of the cannula 60. The shape forming element 69″′ permits the mouthpieces 69′ and/or 69″ to be bent, contoured, configured or molded into a desired shape, configuration and/or position so that the mouthpiece end openings 83, 87 are suitably aligned and located to communicate with air exhaled from the patient's mouth while still retaining such desired shape, configuration or position following adjustment of the mouthpiece 69′ and/or 69″. Shape forming element 69″′ can either be formed integral with the first and/or the second mouthpieces 69′, 69″, can be accommodated within an integral compartment or chamber which extends substantially along the entire length of the first and/or the second mouthpieces 69′ and/or 69″ between the two mouthpieces, as diagrammatically illustrated in FIGS. 16 and 17, or can be glued or otherwise permanently affixed or secured to an exterior surface of either the first and/or the second mouthpieces 69′ and/or 69″, along a substantial portion or length thereof (not shown).

Referring now to FIGS. 18A-18G, seven variations of the nasal cannula, according to the present invention, are shown and still other embodiments would be readily apparent to those skilled in the art. According to these embodiments, the cannula 60 may be provided with two similarly sized mouthpieces 69′ or 69″ (see FIG. 18A where both mouthpieces have a relatively large internal flow passage and FIG. 18G where both mouthpieces have a relatively small internal flow passage), with two differently sized mouthpieces 69′ or 69″ (see FIG. 18E where the left side mouthpiece has a relatively large internal flow passage while the right side mouthpiece has a relatively small internal flow passage and FIG. 18F where the right side mouthpiece has a relatively large internal flow passage while the left side mouthpiece has a relatively small internal flow passage) with only a single mouthpiece 69′ or 69″ (see FIG. 18B where only the first main body chamber 71′ communicates with a mouthpiece which has a relatively large internal flow passage and FIG. 18C where only the second main body chamber 71″ communicates with a mouthpiece which has a relatively large internal flow passage), or without any mouthpiece at all (see FIG. 18D), depending upon the particular application(s), function(s) and/or requirement(s) of the cannula 60. In addition, each of the cannulas 60 may or may not include any shape forming element 69″ and may or may not include one or more alternate flow path opening(s) 35, 37 in either one or both of the nares 65, 67. With respect to the embodiments illustrated in FIGS. 18E, 18F and 18G, for example, at least one of the mouthpieces 69′ and/or 69″ of the nasal cannula 60 has a small external diameter or size and a correspondingly smaller internal flow passage which is suitable for pediatric applications, e.g., suitable for use with the physical size of a pediatric patient where generally lower gas flow volumes and pressures are appropriate or desired.

Next considering further aspects of a nasal cannula 60 of the present invention as illustrated in FIGS. 16 and 17, for example, it will be understood that the volume and the pressure of gas flow from a patient, through the first main body chamber 71′ and the first tube 74, to a gas monitoring/analysis device 86MA is typically less than the volume and the pressure of gas flow from a gas source 86G to the patient, through the second tube 76 and the second main body chamber 71″. It is also preferable to deliver a gas sample from the patient through the first main body chamber 71′ and the first tube 74 as efficiently as possible and with as little reading time delay as is practically possible, and it is understood that a smaller volume of gas at the same, or possibly a slightly lower, supply pressure will propagate more rapidly through a smaller diameter supply tube than through a larger diameter supply tube.

In the embodiments of the cannula 60 illustrated in FIGS. 16 and 17, and for this reason, at least the internal diameters of first tube 74 and of the mating end regions of first main body chamber 71′ are of a reduced size relative to the second tube 76 and the corresponding mating end regions of the second main body chamber 71″, thereby providing a more efficient gas flow sample passageway for the exhaled gases from the patient, at the reduced volumes, but substantially at the same supply pressure. It is also within the spirit and scope of the present invention to reduce the transverse cross section of the first main body chamber 71′ (not shown) thereby providing a more efficient gas flow sample passageway for the exhaled gases from the patient. According to the embodiments of the cannula shown in FIGS. 16 and 17, the internal transverse cross sectional flow passage or diameter of the first tube 74 typically ranges between about 0.0030 and about 0.0010 square inches and more preferably ranges between about 0.0022 and about 0.0017 square inches or so, for example—e.g., the internal diameter of the first tube is typically 0.050+0.003 inches.

For the input side of the cannula 60, however, the internal transverse cross sectional flow passage or diameter of the second tube 76, which communicates with the second main body chamber 71″, typically ranges between about 0.0060 and about 0.0030 square inches and more preferably ranges about 0.0050 and about 0.0038 square inches or so, for example, (e.g., the internal diameter of the second tube is typically 0.075+0.005 inches. and is suitable for conveying a larger volume of gas at the same, or possibly a higher, pressure and, for this reason, the diameter second tube 76 and the mating regions of second main body chamber 71″, are typically of approximately the same diameter as described with regard to the nasal cannula illustrated in FIGS. 1 through 15, for example. For pediatric applications, both of the first and second tubes will typically have an internal transverse cross sectional flow passageway of substantially the same as noted above.

Considering still further aspects of the nasal cannula 60 of the present invention, as represented in FIGS. 16 and 17 and described herein, the alternate path openings 35, 37 of one or more of nares 65, 67 prevent the occurrence of an undesirable level of suction at the nare end openings 62, 64 and also provide one or more secondary flow paths between the patient's nostrils and the first and the second main body chambers 71′ and 71″ in the event that the primary flow paths, through the nare end openings 62, 64, become either partially or fully occluded, blocked, clogged or otherwise obstructed, during use. FIG. 16 illustrates, for example, an embodiment of the nasal cannula 60 where the nare 65 is provided with at least one alternate flow path opening(s) 35 while the nare 67 is also provided with at least one alternate flow path opening(s) 37.

According to the present invention, however, it is recognized and appreciated that the presence of the alternate flow path opening(s) 35, 37, in one of the nares 65 or 67, may possibly interfere with, for example, obtaining an accurate and reliable exhaled gas sample for supply to the monitoring/analysis device and/or for monitoring of the patient's end tidal breathing by a capnagraph device 86MA. For example, the monitoring/analysis device or the capnagraph device 86MA typically will actively withdraw gas samples from the first main body chamber 71′ through the first tube 74. The alternate flow path opening 37 in the nare 65, however, may possibly allow room air to be drawn into the first main body chamber 71′ together with the desired exhaled gas from the patient which is drawn into the first main body chamber 71′ through the nare end opening 62 of the first nare 65 and/or the mouthpiece end opening 83 of mouthpiece 69′, thereby possibly diluting the sample. Also possible, the alternate flow path opening 37 in the nare 65 may allow room air to be drawn into or pushed out of the first main body chamber 71′ as the patient inhales and exhales, thereby possibly reducing the amplitude of and/or distorting the waveform of the end tidal breathing pressure changes that the capnagraph device 86MA is measuring via the first tube 74 and the first main body chamber 71′.

The presence or absence of an alternate flow path opening(s) 35, 37 in the nares 65, 67, therefore, requires consideration of the possible benefits as well as the associated possible disadvantages provided by the alternate flow path opening(s) 35, 37. For example, on the “output” side of the cannula 60, that is, the flow path involving the nare 65 and the first main body chamber 71′, the gas samples are often withdrawn from the first main body chamber 71′ and through the first tube 74 by suction exerted, for example, by monitoring/analysis device 86MA. The suction exerted by monitoring/analysis device 86MA thereby increases the possibility that the nare end opening 62 may be partially or wholly occluded, blocked and/or obstructed, so that the alternate flow path opening 37 in the nare 65 would be beneficial. In opposition, however, and as discussed above, the volume and possibly the pressure of the gas flow drawn by the first main body chamber 71′ is relatively low, thereby reducing the possibility that the nare 65 may become partially or wholly occluded, blocked or obstructed during use. At the same time, the volume of gas drawn into the first main body chamber 71′, for each gas sample, is relatively small so that any contamination or dilution thereof, by room air for example, could be significant and thereby alter accurate measurement of the same.

In the embodiment of the nasal cannula 60 of the present invention illustrated in FIG. 17, therefore, and for the above discussed reasons, the nare 65, which communicates with the first main body chamber 71′ flow path, includes at least one alternate flow path opening 37 to reduce and/or eliminate the risk of partial or complete occlusion, blockage and/or obstruction of the nare 65 and to increase the probability that an adequate gas sample will be supplied to monitoring/analysis device 86MA.

It will be noted, however, that in the embodiment illustrated in FIG. 17, the nare 67, which forms part of the second main body chamber 71″ flow path, e.g., the “input” or supply side of the nasal cannula 60, is not provided with an alternate flow path opening 35 for the reasons discussed above. More specifically, the gas source 86G does not apply any suction or negative pressure to the second main body chamber 71″, unlike the monitoring/analysis device 86MA, but instead provides an insufflation gas, at a positive pressure, through the second tube 76 and to the second main body chamber 71″. In addition, a capnagraph device, or some other similar device, could be utilized as the gas source 86G, e.g., connected to the second main body chamber 71″, either directly or through the second tube 76, measures variations in the gas pressure in the second main body chamber 71″, as the patient inhales and exhales, and thereby, in effect, measures the “back pressure” in the second main body chamber 71″ resulting from the patient's breathing. As a consequence of this, the second main body chamber 71″ is typically at a positive or neutral pressure so that the possibility of the nare end opening 62 becoming partially or completely blocked and/or obstructed by a substances or being attached to the nasal tissue by suction, is minimal so that the alternate flow path opening 35 would provide little benefit. To the contrary, and as discussed above, the alternate flow path opening 35 provides communication with, for example, the room air and such air may have a detrimental effect on the capnagraph measurements as the flow of room pressure air into and out of the second main body chamber 71″ may tend to reduce the amplitude of and/or distort the waveform of the tidal breathing pressure changes that the capnagraph device 86G is measuring through the second tube 76 and the second main body chamber 71″.

For these reasons, therefore, the embodiment of the nasal cannula 60 of the present invention, as illustrated in FIG. 17 for example, does not include any alternate flow path opening 35 in the nare 67.

Referring next to FIGS. 19A-19D, the embodiments of a cannula illustrated therein are very similar to the previously described embodiments, so that only the differences between these embodiments and the prior embodiments will be discussed in detail.

As shown in FIG. 19A, this embodiment is for an undivided nasal cannula 60 which does not include mouthpiece. In addition, each one of the nares 65, 67 includes at least one alternate flow path opening 35, 37 therein. This cannula is suitable for adult, infant and pediatric applications.

As shown in FIG. 19B, this embodiment is for an undivided nasal cannula 60 which also does not include mouthpiece. In addition, neither one of the nares 65, 67 includes any alternate flow path opening therein. This cannula is suitable for adult, infant and pediatric applications.

As shown in FIG. 19C, this embodiment is for an undivided nasal cannula 60 which includes a pair of separate mouthpieces 69′, 69″. If desired, a single. large opening (not shown) can be formed in the free, remote end of the separate mouthpieces 69′, 69″ to form a common inlet/outlet for both of the separate mouthpieces 69′, 69″. In addition, each one of the nares 65, 67 includes at least one alternate flow path opening 35, 37 therein. This cannula is suitable for adult, infant and pediatric applications.

As shown in FIG. 19D, this embodiment is for an undivided nasal cannula 60 which includes a pair of separate mouthpieces 69′, 69″. If desired, a single. large opening (not shown) can be formed in the free, remote end of the separate mouthpieces 69′, 69″ to form a common inlet/outlet for both of the separate mouthpieces 69′, 69″. Neither one of the nares 65, 67 includes any alternate flow path opening therein. This cannula is suitable for adult, infant and pediatric applications.

It is to be appreciated that if a device 86G or 86MA is for either supplying a gas or medication to a patient or detecting breathing or pressure sensing of the patient, then the nare 65 or 67 preferably is not provided with an alternative flow path 35 or 37 therein.

In a preferred form of the invention, the ends of the first and second tubes have the same fittings to facilitate manufacture of the cannula assembly. To further facilitate use of the cannula, the first smaller tube has a first color, e.g., the first tube is clear, while the second larger tube has a different color, e.g., is light green for example, and such color coding facilitates proper connection of the respective tubes with the correct devices 86G and 86MA.

When using the cannula 60 during a typically sleep lab application, one of the tubing 74, 76, e.g., the larger size tubing 76, will be detecting pressure while the patient is sleeping, while the other tubing, e.g., the smaller size tubing 74, will be sampling the amount of CO₂ being exhaled by the patient.

Although the embodiments of FIGS. 16-18H are shown to include a conventional hydrophobic/anti-microbial filter, it is to be appreciated that such filter could be removed without departing from the spirit and scope of the present invention. Further, it is to be appreciated that, although not shown in FIGS. 16-18G of the drawings, a coupling device 78 may be provided for joining or coupling a portion of the second sections of the first and the second conduits or tubings 74′, 76′ to one another, as in the previous embodiments.

It is to be appreciated that the large “green” tube or conduit is particularly adapted for obtaining a desired air flow which is suitable for either pressure monitoring or snore monitoring while the smaller “clear” tube or conduit is particularly adapted for obtaining desired gas samples from the patient. Further, the different type and/or style connector may be provided at each free end of the second sections of the first and the second conduits or tubings 74′, 76′ to facilitate ease of connection of those connectors with the desired device 86G or 86MA. Alternatively, the same type and/or style connector may be provided at each free end of the second sections of the first and the second conduits or tubings 74′, 76′ to facilitate ease of manufacture of the same.

Referring now to FIG. 20, as described above FIG. 20 is a diagrammatic illustration of a cannula 60 specifically adapted for use in the diagnosis of apnea symptoms, such as by polysomnogram. Presently preferred methods for the detection and measurements of apnea and hypopnea employ oral measurements of air flow taken at the mouth for the detection and measurement of apnea episodes and pressure measurements of air flow at the nostrils for the detection and measurement of hypopnea episodes. For this reason, a presently preferred embodiment of an apnea/hypopnea detection cannula 60 has at least one airflow measuring temperature sensor associated with at least one mouthpiece and at least one pressure sensor, and possibly an airflow measuring temperature sensor, associated with at least one nare. As also noted, it will be recognized that the cannula described in and with reference to FIGS. 1-19D may be adapted for use in apnea diagnosis by incorporation of the elements and features of an apnea diagnosis cannula 60 such as that illustrated in FIG. 20.

As illustrated in FIG. 20, a apnea diagnosis nasal cannula 60 of the present invention includes a generally tubular hollow main body 71 that may be internally divided by a partition, a divider or a septum 81 into a first main body chamber 71′ and a second main body chamber 71″. As discussed herein above, the first and the second main body chambers 71′ and 71″ each provide separate gas flow paths between the patient and desired ones of various gas sources and measuring and analysis devices, as also discussed herein above.

For example, as shown in FIG. 20 and a previous discussed with regard to, for example, FIGS. 16 and 17, the first main body chamber 71′ and the first chamber end opening 73 typically serve as an output path from the patient and through a first tube 74 to, for example, an monitoring/analysis device 86MA while the second main body chamber 71″ and the second chamber end opening 75 may serve as an input or supply path to the patient from, for example, a source 86G for an insufflating gas through a second tube 76 that is connected to second chamber end opening 75. It will be understood, however, that for the specific purposes of apnea diagnosis it is necessary only to monitor and measure the flow of gases into and out of the patient while the patient is sleeping. For this reason, the primary purpose of the apnea diagnosis cannula 60 is to mount and support the gas flow sensors in the desired positions at either or both of the patient's nasal and oral passages and to provide, in effect, an “anchor point” for leads running between the sensors and the measuring and recording apparatus, which is indicated generally in FIG. 20 as measurement/recording apparatus 86MR.

It will therefore also be recognized that an apnea diagnosis cannula 60 may or may not be connected to a monitoring/analysis device 86MA or a source 86G; the possibility of such a connection is, however, indicated in FIG. 20. If it is desirable or necessary to connect the cannula 60 to either or both of a monitoring/analysis device 86MA or a source 86G the connections will be similar to those discussed above with other embodiments of a cannula 60. For example, a section of a first conduit or tubing 74 may connected to the first end chamber opening 73 while a section of a first end of a second conduit or tubing 76 may be connected to a second chamber end opening 75. The opposed second ends of the first section of the first and the second conduits or tubings 74 and 76 are typically joined with one another by a conventional coupling device 78 and a second section of the first and the second conduits or tubings 74′, 76′ extends from the coupling device 78 to the desired equipment, apparatus or device 86MA or 86G. The opposite end of the second section of each of the first and the second conduits or tubings 74′, 76′ typically has a luer connector 82′ (e.g., either male of female) which may be either coupled to a filter 84, prior to engaging with the equipment, apparatus or device 86MA or 86G or may be directly couple to the equipment, apparatus or device 86MA or 86G. If a filter is utilized, the filter 84 may be incorporated into the conventional luer connector 82 as this unitary structure facilitates coupling of the nasal cannula 60 to the equipment, apparatus or device 86MA or 86G in a conventional fashion. The first section of each of the first and the second conduits or tubings 74 and 76 may, for example, each have a total length of about 8 inches to about a 24 inches or so and preferably have a length of about 15 to 25 inches or so while the second sections of each of the first and the second conduits or tubings 74′, 76′ each typically have a length of about 3 feet to about 10 feet, preferably a length of about 5 to 7 feet or so.

As also discussed herein above with reference to the embodiments of a cannula 60 illustrated in FIGS. 1-19D, an apnea diagnosis nasal cannula 60 may provide separate gas flow paths between one of the first and the second main body chambers 71′ and 71″ and a desired one of the nasal passages and the mouth of a patient. For example, and as described herein above, the flow passages between the first and the second main body chambers 71′ and 71″ and the nasal passages of the patient are provided by the first and the second nares 65 and 67. The flow path between the first main body chamber 71′ and a first nasal passage of the patient is formed by the internal flow passage 91 through the first nare 65 and the first nare end opening 62 in the first nare 65, while the flow path between the second main body chamber 71″ and the second nasal passage of the patient is formed by the internal flow passage 95 through the second nare 67 and the second nare end opening 64 in second nare 67. As has been described herein above, the flow path 95 through the second nare 67 may, for example, convey a flow of insufflating gas, e.g., oxygen, from the gas source 86G to the patient, while the flow path 91, through the first nare 65 may, for example, convey gas sample(s) from the patient to the gas monitoring/analysis devices 86MA.

Further flow paths, between the first and the second main body chambers 71′ and 71″ and a patient's mouth, are also provided by first and second mouthpieces 69′ and 69″ which, as described herein above, are essentially integral hollow tubes extending in a curved configuration downward from the main body 71 and communicating with either the first or the second main body chambers 71′ and 71″ and terminating or ending at or in the region in front of the patient's mouth. The first mouthpiece 69′, for example, includes a first internal flow path 77 communicating with and extending from the first main body chamber 71′ and terminating in a mouthpiece end opening 83 which is positionable adjacent an open mouth of the patient. The second mouthpiece 69″, in turn, includes a second internal flow path 79 communicating with and extending from the second main body chamber 71″ and terminating in a mouthpiece end opening 87 which is positionable adjacent an open mouth of the patient. It will be understood that the second mouthpiece 69″ may, for example, provide a flow path, for example, for the insufflating gas to the patient while the first mouthpiece 69′ may, for example, provide a flow path by which a gas sample, for example, may be retrieved from the patient's exhaled breath and conveyed to the gas monitoring/analysis devices 86MA.

As also discussed herein above, one or both of the mouthpieces 69′, 69″ of the cannula 60 can be provided with a shape forming and retaining element 69″ that may comprise, for example, a shape retaining, dead soft pliable material, element, component or wire, such as copper, to facilitate desired alignment and/or retention of the first and/or the second mouthpieces 69′, 69″ in a desired aligned orientation or position, relative to the patient's mouth during use of the apnea diagnosis nasal cannula 60. The shape forming element 69″ permits the mouthpieces 69′ and/or 69″ to be bent, contoured, configured or molded into a desired shape, configuration and/or position so that the mouthpiece end openings 83, 87 are suitably aligned and located to communicate with air exhaled from the patient's mouth while still retaining such desired shape, configuration or position following adjustment of the mouthpiece 69′ and/or 69″. Shape forming element 69″′ can either be formed integral with the first and/or the second mouthpieces 69′, 69″, can be accommodated within an integral compartment or chamber which extends substantially along the entire length of the first and/or the second mouthpieces 69′ and/or 69″ between the two mouthpieces, as diagrammatically illustrated in FIGS. 16 and 17, or can be glued or otherwise permanently affixed or secured to an exterior surface of either the first and/or the second mouthpieces 69′ and/or 69″, along a substantial portion or length thereof (not shown).

It will be recognized that, given that the primary purpose of an apnea diagnosis nasal cannula 60 is to position and support one or more sensors with respect to a patient's nasal and oral passages, there may be a number of variant embodiments of an apnea diagnosis nasal cannula 60. For example, while an apnea diagnosis nasal cannula 60 will typically include a main body 71, the main body 71 may not be divided into first and second main body chambers 71′ and 71″ septum 81. In a like manner, while an apnea diagnosis nasal cannula 60 will typically include first and second nares 65, 67, the cannula may include only a single mouthpieces 69′ or 69″. The following description of an apnea diagnosis nasal cannula 60 of the present invention will, however, refer to the apnea diagnosis nasal cannula 60 as an exemplary embodiment of the present invention.

As discussed previously, the purpose and function of an apnea diagnosis nasal cannula 60 is to detect and measure air flows and pressures in both apnea and hypopnea wherein, as also discussed, apnea and hypopnea are distinguished in that hypopnea results in a reduced but continuing air flow while apnea, results in a cessation of air flow and is accompanied by a lack of chest movement. As also discussed, the location at which air flow is detected, that is, at the nasal passages or at the oral passage, and the types of sensors used at each location for detecting apnea and hypopnea are significant factors. For example, in a presently preferred embodiment of an apnea diagnosis nasal cannula 60 apnea episodes are preferably detected by means of mouthpiece measurements, that is, measurements of the oral passage airflow, using thermal sensors such as thermistors or thermocouples, while it is presently preferred to rely on measurements taken by pressure sensors at the nasal passages to detect hypopnea episodes.

As discussed herein above, the present invention is directed to an apnea diagnosis nasal cannula 60 having an at least one airflow measuring temperature sensor associated with at least one mouthpiece and having either or both of a pressure sensor and an airflow measuring temperature sensor associated with at least one nare for the purpose of detecting, measuring and diagnosing patient apnea and/or hypopnea. Therefore referring again to FIG. 20, the exemplary embodiment of an apnea diagnosis nasal cannula 60 illustrated therein includes at least one nasal sensor 200N located within at least one nare internal flow passage, with the exemplary embodiment shown in FIG. 20 having a nasal sensor in each nare flow passage for purposes of description and discussion. In the exemplary embodiment illustrated in FIG. 20 there is a first nasal sensor 200′ located in nare flow path 91 that passes through first nare 65 between first nare end opening 62 and first main body chamber 71′ and a second nasal sensor 200″ located in nare flow path 95 that passes through second nare 67 between second nare end opening 64 and second main body chamber 71″. As indicated, nasal sensors 200′, 200″ are located within nare flow passages 91 and 95 to be in the airflow path to and from the corresponding patient nasal passage, and are located in nare passages 91 and 95 in a region close to the main body 71, that is, in the region of greatest width of nare flow passages 91 and 95, to present a minimum obstruction to the air flow through nare flow passages 91 and 95.

As illustrated in FIG. 20, a nasal sensor 200′ or 200″ may be mounted on the inside surface of their respective nares 65, 67, as is illustrated in FIG. 20 for the case of nasal sensor 200′. In this case, again as illustrated by the example of nasal sensor 200′ in FIG. 20, the read 202′ or 202″ of a nasal sensor 80′ or 200″ may be lead along the inside surfaces of the respectively nare flow passage 91 or 95 and the inside surfaces of the corresponding first and second main body chamber 71′ or 71″ to end opening 73 or 75, or to pass-through openings in the region of the end opening 73 or 75, for connection by appropriate leads or wiring to measurement/recording apparatus 86MR.

In other embodiments, however, such as illustrated for the case of nasal sensor 200″ in FIG. 20, the nasal sensor 200′ or 200″ may be mounted in a sensor opening 104 extending through the wall of the respective nare 65 or 67 so that the nasal sensor 200′ or 200″ extends into the corresponding nare flow path 91 or 95. The leads 202′ or 202″ of the nasal sensor 80′ or 200″ will be accessible on the outer ends of the opening 204 and may be lead along the outside surfaces of the corresponding nare 65 or 67 and first and second main body chambers 71′ or 71″ to the outer ends of the main body chambers 71′ or 71″ for connection by appropriate leads or wiring to measurement/recording apparatus 86MR.

The exemplary embodiment of an apnea diagnosis nasal cannula 60 illustrated in FIG. 20 further includes at least one oral sensor 206′ or 206″ located within the internal flow path 77 or 79 of at least one mouthpiece 69′ or 69″. The exemplary embodiment of FIG. 20 is shown as having an oral sensor 206′ or 206″ in the internal flow path 77 or 79 each of mouthpieces 69′ and 69″ for purposes of description and discussion.

As in the case of nasal sensors 200′, 200″, an oral sensor 206′ or 206″ may be mounted on the inside surface of their respective mouthpiece 69′ or 69″, as is illustrated in FIG. 20 for the case of oral sensor 206′. In this case, and as illustrated for oral sensor 206′, the lead 208′ or 208″ of the oral sensor 206′ or 206″ may be lead along the inside surface of the respectively internal flow path 77 or 79 and the inside surfaces of the corresponding first and second main body chamber 71′ or 71″ to the end opening 73 or 75, or to pass-through openings in the region of the end openings 73 or 75, for connection by appropriate leads or wiring to measurement/recording apparatus 86MR.

In the alternative, the oral sensor 206′ or 206″ may again be mounted in a sensor opening 98 extending through the wall of the respective mouthpieces 69′ or 69″ so that the oral sensors 206′ or 206″ extends into the internal flow path 77 or 79. The lead 208′ or 208″ of the oral sensor 206′ or 206″ will be accessible on the outer ends of the sensor openings 210 and may be lead along the outside surface of the corresponding mouthpiece 69′ or 69″ and the corresponding first or second main body chambers 71′ or 71″ to the outer end of the corresponding main body chamber 71′ or 71″ for connection by appropriate leads or wiring to measurement/recording apparatus 86MR.

In yet a further alternative, the sensors may be mounted into the cannula 60 by means of clips or other “forms of holders or brackets, possible comprised of the same materials as the body of the cannula 60.

Next considering nasal sensors 200′, 200″ and oral sensors 206′, 206″, as described previously the two primary types of airflow sensor used for polysomnograms are thermistor and thermocouple type sensors that detect airflow and volume by changes in temperature caused by the air flow and pressure sensors that detect air flow and volume by pressure changes resulting from air flow. Either type of sensor may be employed as an nasal sensor 200′, 200″ or as an oral sensor 206′, 206″, with the selection being a function of, for example, the sensitivity, accuracy, response times, physical size, wiring requirements and costs of the sensors. For example, a thermal type sensor such as a thermistor or thermocouple will typically be smaller and less expensive than a pressure sensor and will require fewer wiring connections, thereby reducing the number of leads running through or on the body, nares and mouthpieces of the apnea diagnosis nasal cannula 60 and the connections to a measurement/recording apparatus 86MR. A thermal type sensor, however, essentially measures the cooling effect of the flow of air around the sensor and is typically slower to react to changes in air flow but may be better able to detect lower levels of air flow because of the greater amplitude of the output signal. A pressure type sensor, however, may respond more quickly to changes in a patient's breathing patterns because the sensor measures air pressure rather than detecting changes in the volume of air flow by its thermal effects, but may be less sensitive to low levels of air flow and thus lower levels of pressure change.

In a presently preferred embodiment of an apnea diagnosis nasal cannula 60, therefore, the oral sensor or sensors 206′, 206″ are comprised of thermal type sensors, that is, thermistor or thermocouple type sensors, while nasal sensor or sensors 200′ and 200″ are preferably comprised of pressure type sensors with possible a thermal type sensor as an auxiliary or backup sensor, such as a pressure type sensor in one nare 65, 67 and a thermal type sensor in the other nare 65, 67.

Lastly, it will be recognized that, as described previously, other implementations of an apnea diagnosis nasal cannula 60 may be employed and may include cannula having, for example, a main body 71 that is not divided into first and second main body chambers 71′ and 71″ by a septum 81 but instead has a single body chamber linking both nares and the mouthpieces, if any. In a like manner, while an apnea diagnosis nasal cannula 60 will typically include first and second nares 65, 67, the cannula may include only a single mouthpieces 69′ or 69″. In addition, other implementations of an apnea diagnosis nasal cannula 60 may include, for example, multiple sensors in the nares or mouthpieces or a sensor in only one nare rather than both.

Since certain changes may be made in the above described improved cannula and method of using the same, without departing from the spirit and scope of the invention herein involved, it is intended that all of the subject matter of the above description or shown in the accompanying drawings shall be interpreted merely as examples illustrating the inventive concept herein and shall not be construed as limiting the invention. That is, the invention described herein is to be limited only by the scope of the appended claims and the applicable prior art.

I/We claim:

1. A nasal cannula for monitoring breathing of a patient, the nasal cannula comprising:
an elongated main body for positioning adjacent a nose of the patient and having at least one main body chamber communicating with a first cannula inlet/outlet and a second cannula inlet/outlet;
a first nare formed integral with and extending from the main body chamber of a length and a size for being received within a first nasal passage of a patient's nose and forming a first gas flow passage extending from a inlet/outlet opening formed in an end surface of the first nare and the main body chamber,
a second nare formed integral with and extending from the main body chamber of a length and a size for being received within a second nasal passage of a patient's nose and forming a second gas flow passage extending from a inlet/outlet opening formed in an end surface of the second nare and the main body chamber,
at least one mouthpiece having a first end at the main body chamber and a second end positionable at a mouth of the patient and having a mouthpiece gas flow passage extending between the main chamber and a mouthpiece inlet/outlet at the second end of the mouthpiece,
a least one nasal gas flow sensor located in an gas flow passage of at least one of the first and second flares, and
at least one oral gas flow sensor located in the mouthpiece gas flow passage.

2. The nasal cannula of claim 1, wherein:
the at least one nasal gas flow sensor is located in the gas flow passage adjacent the main body chamber.

3. The nasal cannula of claim 1, wherein:
the oral gas flow sensor is located in the mouthpiece gas flow passage adjacent the main body chamber.

4. The nasal cannula of claim 1, further comprising:
a first nasal gas flow sensor located in first gas flow passage of the first nare, and
a second nasal gas flow sensor located in the second gas flow passage of the second nare.

5. The nasal cannula of claim 1, wherein the at least one mouthpiece comprises:
a first mouthpiece having a first oral gas flow sensor located in a first mouthpiece gas flow passage adjacent the main body chamber, and
a second mouthpiece having a second oral gas flow sensor located in a second mouthpiece gas flow passage adjacent the main body chamber.

6. The nasal cannula of claim 1, further comprising:
a septum dividing the main body chamber into a first main body chamber communicating with the first cannula inlet/outlet and the first gas flow passage of the first nare and a second main body chamber communicating with the second cannula inlet/outlet and the second gas flow passage of the second nare and wherein the mouthpiece gas flow passage of the at least one mouthpiece communicates with one of the first and second main body chambers.

7. The nasal cannula of claim 5, further comprising:
a septum dividing the main body chamber into a first main body chamber and a second main body chamber, wherein
the first main body chamber communicates with the first cannula inlet/outlet and the first gas flow passage of the first nare and the second main body chamber communicates with the second cannula inlet/outlet and the second gas flow passage of the second nare, and wherein
the first mouthpiece gas flow passage of the first mouthpiece communicates with the first main body chamber and the second mouthpiece gas flow passage of the second mouthpiece communicates with the second main body chamber.

8. The nasal cannula of claim 1, wherein:
the at least one nasal gas flow sensor is one of a thermal gas flow sensor and a pressure sensor, and
the at least one oral gas flow sensor is a thermal gas flow sensor.

9. The nasal cannula of claim 4, wherein:
one of the first and second nasal gas flow sensors is a pressure sensor and another of the first and second gas flow sensors is a thermal gas flow sensor.
